(19) United States
(12) Patent Application Publication
(10) Pub. No.: US 2007/0160684 A1
(43) Pub. Date: Jul. 12, 2007

(54) Methods for increasing cancer cell chemosensitivity

(76) Inventors: Yun Yen, Arcadia, CA (US); David Ann, Arcadia, CA (US)

Correspondence Address: PERKINS COIE LLP, POST OFFICE BOX 1208, SEATTLE, WA, 98111-1208, US

(21) Appl. No.: 11/496,014

(22) Filed: Jul. 28, 2006

Related U.S. Application Data
(60) Provisional application No. 60/703,731, filed on Jul. 29, 2005.

(51) Int. Cl. A61K 31/704; C12Q 1/68; A61K 31/70; A61N 5/00

(52) U.S. Cl. 424/649; 514/34; 435/6; 600/1

(57) ABSTRACT

The expression of HMGA2 is often associated with neoplastic transformation. The present disclosure establishes a correlation between increased HMGA2 expression and enhanced chemosensitivity towards DNA-damaging therapeutic agents such as doxorubicin, cisplatin, and X-ray irradiation.

### CROSS-REFERENCE TO RELATED APPLICATIONS

The present utility applications claims priority to U.S. Provisional Application No. 60/703,731, filed Jul. 29, 2005, the disclosure of which is incorporated by reference herein in its entirety.

### GOVERNMENT INTEREST

This invention was made with government support in part by grants from the National Institutes of Health, specifically R01 DE 10742 and DE 14183 (to DKA) and R01 CA 72767 (to YY). The government may have certain rights in this invention.

### BACKGROUND

High-mobility group A2 (HMGA2) protein, also known as HMGI-C, is a non-histone architectural transcription factor belonging to the HMGA family. In addition to HMGA2, the HMGA family consists of HMGA1a, HMGA1b, and HMGA1c (Reeves 2001a). All HMGA proteins contain about 100 amino acid residues and have three DNA-binding domains (AT-hooks) that interact with the narrow minor groove of AT-rich DNA sequences (Reeves 2001 b). Amino acid residues 44 to 68 of HMGA2, located in the second AT-hook, and its corresponding region within HMGA1 have been reported to be critical to proper HMGA1/2 function (Abdulkadir 1995; Chau 2000; Chin 1998; Zhang 1999; Noro 2003; Pierantoni 2005; Fedele 2006). HMGA proteins have been demonstrated to organize the assembly of nucleoprotein transcriptional complexes by binding to DNA and/or transcription factors and enhancing or repressing transcription (Mantovani 1998; Munshi 2001; Reeves 2001 b; Zentner 2001).

HMGA2 expression occurs primarily during embryogenesis, and is below detectable levels or completely absent in differentiated cells from normal adult tissues (Zhou 1995; Zhou 1996; Hirning-Folz 1998). Consistent with its growth regulatory role is the pygmy phenotype in mice in which the HMGA2 gene is homozygously inactivated (Zhou 1995; Anand 2000).

HMGA2 has been implicated in promoting tumorigenesis (Rogalla 1997; Tallini 1999; Fedele 2001; Sgarra 2004). There is a strong correlation between ectopic HMGA2 expression and malignant tumor phenotypes such that HMGA2 overexpression in cancer cells is associated with poor prognosis and high tumor grades (Abe 2003; Langelotz 2003; Tessari 2003; Miyazawa 2004). HMGA2 is disrupted and aberrantly expressed in many tumor types, making it one of the most frequently rearranged genes in human neoplasias (Ashar 1995; Fejzo 1995; Schoenmakers 1995; Tallini 1999). The majority of breaking points in the HMGA2 gene lead to the removal of its acidic C-terminus tail, which is a characteristic feature not only of the HMGA family but also of all HMG family members, including HMGB1, HMGB2, HMGN1, and HMGN2 (Noro 2003). Removal of this acidic tail has been shown to modestly increase affinity of HMGA1 to DNA and to consistently enhance negative supercoiling (Nissen 1995), while having no effect on binding of HMGA1 to the nucleosome (Reeves 1993). Interestingly, the C-terminus tail of HMGA1 and HMGA2 has no activating or repressing activity when fused to the DNA-binding domain of GAL4 (Thanos 1992; Zhou 1996).

HMGA2 expression is correlated with malignant phenotype of mesenchymal or epithelial origin (Finelli 2002; Abe 2003; Masciullo 2003). Transfection of normal rat thyroid cells with an antisense molecule against HMGA2 prevents neoplastic transformation induced by myeloproliferative sarcoma virus and Kristen murine sarcoma virus (Berlingieri 1995). HMGA2 expression in oral squamous cell carcinoma is associated with increased disease recurrence and metastasis, along with a reduced survival rate manifested by a facilitated epithelial-mesenchymal transformation (Miyazawa 2004). In addition, HMGA2 expression in breast cancer patients correlated with poor prognosis and metastasis (Rogalla 1997; Langelotz 2003).

### SUMMARY

In certain embodiments, methods are provided for predicting the likelihood that treatment with a DNA-damaging therapeutic agent will be effective in a subject with cancer by measuring HMGA2 expression in one or more cancer cells from said subject.

In certain embodiments, methods are provided for identifying a subject with cancer who is likely to respond favorably to treatment with a DNA-damaging therapeutic agent by measuring HMGA2 expression levels in one or more cancer cells from the subject. A subject is likely to respond favorably when one or more of these cancer cells exhibits HMGA2 expression levels at or above a threshold level.

In certain embodiments, methods are provided for treating cancer in a subject by measuring HMGA2 expression levels in one or more cancer cells from the subject, then administering one or more DNA-damaging therapeutic agents if one or more of these cells exhibit HMGA2 expression levels greater than or equal to a threshold value.

In certain embodiments, a kit is provided for determining the likelihood that a subject with cancer will respond favorably to treatment with one or more DNA-damaging therapeutic agents. In certain embodiments, such a kit includes a means for measuring HMGA2 expression levels in one or more cancer cells from said subject.

In certain embodiments, methods are provided for increasing the sensitivity of a cancer cell to treatment with a DNA-damaging therapeutic agent by introducing into the cell a polynucleotide encoding HMGA2 or a fragment thereof.

In certain embodiments, methods are provided for increasing the effectiveness of treatment with a DNA-damaging therapeutic agent in a subject with cancer. In certain of these embodiments, the subject is administered a therapeutically effective amount of an HMGA2 polypeptide or a fragment thereof.

In addition to the exemplary embodiments described above, further embodiments and aspects will become apparent by reference to the drawings and by study of the following descriptions.

### BRIEF DESCRIPTION OF THE DRAWINGS

FIG. 1: Schematic of the role of HMGA2 in enhancing cytotoxicity of DNA-damaging therapeutic agents.

FIG. 2: A. Effect of HMGA2 expression on Dox- and irinotecan-mediated growth inhibition in Pa-4 and Pa-4/HMGA2 cells. Cells were incubated with varying concentrations of Dox (upper panel) or irinotecan (lower panel), and cell survival was measured using the MTT cell viability assay (* P<0.05). The percentage of cell viability at each treatment concentration is based on the mean±S.D. of three independent experiments performed in triplicate. Lower panel inset: expression of HMGA2 in Pa-4 (Lane 1) and Pa-4/HMGA2 (Lane 2) cells. B. HMGA2 expression in HCC1419 and HS578T cells. Steady-state HMGA2 expression levels were measured by RT-PCR. GADPH expression levels were measured as a control. C. Effect of HMGA2 expression on Dox-mediated growth inhibition in HS578T, HCC1419, and CHO cells. Cells were incubated with varying concentrations of Dox, and cell survival was measuring using by MTT assay. Statistical analysis was done with HS578T and HCC1419 pair (* P<0.05).

FIG. 3: A. Effect of HMGA2 expression on Dox-mediated G₂/M arrest. Synchronized Pa-4 and Pa-4/HMGA2 cells were treated with Dox or vehicle only for 24 hours. The number of cells in G₁ and G₂/M phase was determined by FACS analysis. The percentage of cells in each phase is summarized in panel e. B. Effect of HMGA2 expression on topo IIα levels. HS578T cells were treated with Dox or vehicle only, and steady-state topo IIα levels were measured by Western analysis. Actin expression levels were measured as a control.

FIG. 4: A. Relationship between Dox treatment and ATM phosphorylation. Upper panel: ATM-deficient pEBS7 and ATM-proficient YZ5 cells were treated with Dox or vehicle only, and ATM phosphorylation was measured by Western analysis. Lower panel: Cell viability was measured by MTT assay. B. Effect of HMGA2 expression on Dox-mediated ATM phosphorylation. Pa-4 and Pa-4/HMGA2 cells were treated with Dox or vehicle only, and ATM phosphorylation was measured by Western analysis. C. Effect of HMGA2 expression on Dox-mediated H2AX phosphorylation. Pa-4 and Pa-4/HMGA2 cells were treated with Dox or vehicle only, and H2AX phosphorylation was measured by Western analysis. D. Panel 1: Effect of HMGA2 expression on Dox-mediated growth inhibition in HeLa and HeLa/HMGA2 cells. Cells were incubated with varying concentrations of Dox, and cell survival was measured using the MTT cell viability assay (** P<0.01). Panel 2: Effect of HMGA2 expression on topo IIα levels. HeLa and HeLa/HMGA2 cells were treated with Dox or vehicle only, and steady-state topo IIα levels were measured by Western analysis. Tubulin expression levels were measured as a control. Panel 3: Effect of HMGA2 expression on Dox-mediated ATM phosphorylation. HeLa and HeLa/HMGA2 cells were treated with varying concentrations of Dox, and ATM phosphorylation was measured by Western analysis. Panel 4: Effect of HMGA2 expression on Dox-mediated H2AX phosphorylation. HeLa and HeLa/HMGA2 cells were treated with varying concentrations of Dox, and H2AX phosphorylation was measured by Western analysis. Actin expression levels were measured as a control.

FIG. 5: A. Effect of caffeine and NU7026 on H2AX phosphorylation in Pa-4 and Pa-4/HMGA2 cells. Cells were pre-treated with vehicle and either caffeine or NU7026, then treated with Dox, caffeine, and NU7026 for 2 hours. H2AX phosphorylation was measured by Western analysis. Actin expression levels were measured as a control. B. Effect of caffeine and NU7026 on H2AX phosphorylation in HS578T and HCC1419 cells. Cells were pre-treated with vehicle and either caffeine or NU7026, then treated with Dox, caffeine, and NU7026. H2AX phosphorylation was measured by Western analysis. Actin expression levels were measured as a control. C. Effect of caffeine on Dox-mediated G₂/M accumulation. Pa-4 and Pa-4/HMGA2 cells were pre-treated with vehicle or caffeine, then treated with Dox and caffeine. The number of cells in G₁ and G₂/M phase was determined by FACS analysis. D. Effect of HMGA2 expression on Dox- and caffeine-mediated apoptosis.

FIG. 6: Effect of Dox treatment on H2AX phosphorylation in pEBS7, YZ5, MO59J, and U2OS.GK41 cells. Upper panel: Cells were treated with Dox, and H2AX phosphorylation was measured by Western analysis. Lower panel: Cells were pre-treated with doxycycline (“induced”) or vehicle only (“non-induced”), then treated with Dox. H2AX phosphorylation was measured by Western analysis. B. Effect of HMGA2 silencing on basal H2AX phosphorylation. HS578T cells were transfected with HMGA2 siRNA, scrambled siRNA, or vehicle only. At various timepoints following transfection, H2AX phosphorylation and HMGA2 expression were measured by Western analysis. Actin expression was measured as a control. C. Effect of HMGA2 silencing on Dox-mediated growth inhibition. HeLa/HMGA2 and HS578T cells were transfected with HMGA2 siRNA, scrambled siRNA, or vehicle only, then treated with various concentrations of Dox. Cell viability was measured by MTT assay (*P<0.05).

FIG. 7: A. Effect of HMGA2 expression on cisplatin-mediated growth inhibition. HeLa, HeLa/HMGA2, HCC1419, and HS578T cells were treated with various concentrations of cisplatin, and cell viability was measured by MTT assay (*P<0.05). B. Effect of HMGA2 expression on X-ray irradiation-mediated growth inhibition. HeLa, HeLa/HMGA2, and HS578T cells were treated with various levels of X-ray radiation, and cell viability was measured using a clonogenic assay (*P<0.05; **P<9.01).

FIG. 8: Association of stable HMGA2 expression with increased DNA damage and dysregulated DNA damage response. A. Plasmid end-joining assays were performed using a pGL2 plasmid containing HindIII and EcoRI restriction enzyme sites (left panel). Relative end-joining efficiency was calculated by comparing luciferase activities of HindIII- or EcoRI-digested pGL2 DNA with that of uncut pGL2, after normalization with pRL-TK for transfection efficiency (right panel. Results shown represent three independent transfection experiments. * denotes p<0.05; ** denotes p<0.01. B. Pa-4 and Pa-4/HMGA2 cells were treated with 5 μM Dox for 2 hours and subjected to Comet assays. DNA damage was quantified by examining 70-100 cells in each set of experiments. The average Olive Tail moment is shown in panel e.

FIG. 9: Association of stable HMGA2 expression with increased DNA damage and dysregulated DNA damage response. A. HMGA2-expressing (Hep3B, HepG2) and HMGA2-under-expressing (CL48) cells were treated with increasing concentrations of Dox (upper panel) or cisplatin (lower panel) for 72 hours. Cell viabilities were assessed using MTT assays. * denotes p<0.05; ** denotes p<0.01. B. HeLa and HeLa/HMGA2 were treated with increasing concentrations of Taxol for 72 hours. Cell viabilities were assessed using MTT assays.

FIG. 10: Association of HMGA2-dependent chemosensitization with DNA-PKcs phosphorylation. Equal amounts of protein lysates prepared from vehicle- or Dox-treated parental or engineered HeLa cells were subjected to Western analyses with anti-phospho-DNA-PKcs-T2609/S2056 and anti-DNA-PKcs antibodies, respectively.

FIG. 11: Association of HMGA2-dependent chemosensitization with DNA-PKcs phosphorylation. HMGA2-expressing Hs578T and HMGA2 under-expressing HCC1419 breast cancer cells were treated with increasing concentrations of Dox. Equal amounts of protein lysates were subjected to Western analyses with anti-HMGA2, anti-phospho-DNA-PKcs-T2609/S2056 and anti-DNA-PKcs antibodies, respectively. For siRNA experiment (lower panel), Hs578T cells were transfected with si-HMGA2 (H) or control siRNA (S) for 48 hours prior to the treatment of Dox.

FIG. 12: Effect of HMGA2 expression on accumulation of pT2609-DNA-PKcs nuclear foci in Dox-treated cells. Representative confocal images of DNA-PKcs-T2609 foci (green) and Dox (red) distribution in HeLa, HeLa/HMGA2, HeLa/HMGA2(2K/R) cells at 30 min post-Dox(5 μM)-treatment (upper panel). The average volume of each DNA-PKcs-T2609 foci in approximate 75-100 cells was quantified after reconstruction in each cell lines and expressed as mean foci volume (panel j). The average number of foci per cell was calculated in 75-100 cells and expressed as mean foci number per cell (panel k).

FIG. 13: Diagram of the functional domains and mutation sites of HMGA2. The shaded boxes represent the AT-hook DNA-binding domain. Mutated amino acid residues in each construct are shown.

FIG. 14: Association of HMGA2-dependent chemosensitization with HMGA2 SUMOylation. HeLa, HeLa/HMGA2, HeLa/HMGA2(2K/R) cells were subjected to treatment with increasing concentrations of Dox (upper panel) or cisplatin (lower panel) for 72 hours, as indicated. Cell viabilities were assessed using MTT assays. * denotes p<0.05; ** denotes p<0.01.

FIG. 15: Association of DNA-PK deficiency with increased chemosensitization to DNA damage agents. Ku70-deficient MEF cells and their wild type counterparts (upper and middle panels) were subjected to treatment with increasing concentrations of Dox (upper panel) or cisplatin (middle panel) for 72 hours. Similarly, DNA-PKcs-deficient (MO59J) and DNA-PKcs-proficient (MO59K) cells were subjected to increasing concentrations of Dox (lower panel). Cell viabilities were assessed using MTT assays. * denotes p<0.05; ** denotes p<0.01.

FIG. 16: Dynamic interaction between HMGA2 and Ku80/Ku70. A. The interaction of HMGA2 and Ku70/80 in HeLa/HMGA2 cells was measured by subjecting equal amounts of protein lysates to co-immunoprecipitation assays and Western analyses, as indicated. B. Ku80 and Ku70 proteins were individually translated in vitro and subjected to His-pulled-down using bacteria-expressed His-tagged HMGA2 as bait.

FIG. 17: Effect of Dox treatment on the HMGA2/Ku70/Ku80 complex. Equal amounts of cell lysates from vehicle- or Dox-treated (A) HeLa and HeLa/HMGA2 cells or (B) Hs578T cells were subjected to reciprocal co-immunoprecipitation assays followed by Western analyses, as indicated. IgG_{HC}: IgG heavy chain; IgG_{LC}: IgG light chain.

FIG. 18: Role of protein-protein and protein-DNA interactions in the HMGA/Ku80/Ku70 interaction. Individual wild type and engineered HMGA2 expression constructs was transfected into HeLa cells. The transfected cells were allowed to recover overnight in fresh medium prior to immunoprecipitation with an anti-HA antibody. Quantitation was done with BioRad Quantity One volume report program. After normalization with transfection efficiency and the expression level of HMGA2 and its mutant, percentage of Ku80/HMGA2 interaction is calculated by designating immunoprecipitated Ku80 with wild type HMGA2 as 100%.

FIG. 19: Correlation between HMGA2 binding of Ku80 and chemosensitization towards Dox. HeLa cells were transfected with individual HMGA2 wild type and engineered constructs and allowed to recover overnight prior to reseeding for cell viability assay. Cells were treated with Dox for 72 hours and followed by MTT assays. ** denotes p<0.01. The expression levels of transfected HMGA2 and its variants in HeLa cells utilized for MTT assays are showed in the lower panel.

FIG. 20: Identification of downstream molecules involved in Dox/HMGA2-mediated chemosensitization. HeLa, HeLa/HMGA2 and HeLa/HMGA2(2K/R) cells (A and C) and MO59J and MO59K (B) cells were treated with Dox (2 μM) for different periods. Equal amounts of protein lysates from vehicle or Dox-treated cells were subjected to Western analyses with anti-phospho-ATM and anti-phospho-Chk2(Thr-68, pThr-68-Chk2), anti-Chk2, anti-phospho-p53 (Ser-15, pSer-15-p53), respectively, as indicated.

FIG. 21: HMGA2 facilitation of Dox-induced caspase-3 activation and p53 expression. A. Correlation between HMGA2-mediated chemosensitization and caspase-3 activations. Equal amounts of protein lysates from different Dox-treated HMGA2-expressing and under-expressing cells were subjected to Western analyses with anti-caspase-3 and anti-p53 antibodies, respectively. The steady-state level of tubulin was used as a loading control. CF: cleaved fragment.

FIG. 22: Effect of HMGA2 expression Dox-induced PUMA and NOXA transcriptional activation. Equal amounts of total RNA prepared from cells treated with 2 μM Dox for different time periods were subjected to real-time RT-PCR analyses to assay the time-dependent transcriptional activations of p53-targeted genes, PUMA and NOXA. Fold induction is calculated using 2^{-ΔΔCt} method, and the average of two independent experiments is shown.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

### Abbreviations

The following abbreviations are used herein: ATM, ataxia-telangiectasia mutated kinase; ATR, ATM- and Rad3-related kinase; ATR-kd, ATR dominant negative; DNA-PK, DNA-dependent protein kinase; DNA-PK, DNA-PK catalytic subunit; Dox, doxorubicin; DSB, double-strand break; HMGA2, high-mobility group A2 protein; NHEJ, non-homologous end joining; PI 3-K, phosphatidylinositol 3-kinase; PIKK, phosphatidylinositol 3-kinase-related kinase; topo, topoisomerase.

To analyze the effects of ectopic HMGA2 expression on cellular response to extracellular insults, Pa-4 and Pa-4/HMGA2 cells were treated with Dox or irinotecan, and cell viability was measured using MTT assays. Dox is an anthracycline chemotherapeutic agent, which functions in part by stabilizing the topo 1′-DNA complex and resulting DSBs upon colliding with the replication fork (Li 2001). Irinotecan is a topo I inhibitor. As expected, treatment with Dox or irinotecan decreased cell viability in both cell types in a dose-dependent manner. The decrease in cell viability generated by irinotecan treatment was essentially the same in both the presence and absence of HMGA2 expression. However, the decrease in cell viability generated by Dox treatment was substantially larger in cells expressing HMGA2, suggesting that HMGA2 enhances Dox-induced cytotoxicity.

If HMGA2 expression were primarily responsible for the enhanced chemosensitivity to Dox that was observed in Pa-4/HMGA2 cells, it would be expected that other HMGA2-overexpressing cells would exhibit similar chemosensitivity to Dox treatment. To determine if this was indeed the case, the following cell lines were examined: HeLa, HeLa/HMGA2, the breast cancer cell lines HS578T and HCC1419, which each display different levels of HMGA2 expression, and HMGA2-deficient CHO cells. Endogenous HMGA2 expression was measured in HS578T and HCC1419 cells, revealing that HMGA2 expression levels were higher in HS578T cells. Each cell type was treated with varying concentrations of Dox, and cell viability was measured by MTT assay. All five cell types showed decreased viability in the presence of Dox, but the level of this growth inhibition varied between cell types. Dox-mediated genotoxicity was more pronounced in HeLa/HMGA2 cells than HeLa cells, and more pronounced in HS578T cells than it was in HCC1419 or CHO cells. These results support the notion that increased levels of HMGA2 are associated with increased responsiveness to Dox treatment.

Dox delays the G₂/M transition (Mikhailov 2004). To examine the mechanism by which HMGA2 enhances Dox cytotoxicity, experiments were performed to determine whether HMGA2 enhances Dox-mediated G₂/M arrest. Pa-4 and Pa-4/HMGA2 cells were treated with either Dox or vehicle only, and the percentage of cells in various stages of the cell cycle was determined by FACS analysis. As expected, treatment with Dox greatly increased the percentage of cells in G₂/M phase. Apoptosis of Pa-4 and Pa-4/HMGA2 cell was quantified by assessing the sub-G₁ cell population, a hallmark of cell apoptosis. Dox increased apoptosis levels in Pa-4/HMGA2 almost five-fold, versus an increase of only two-fold in Pa-4 cells.

These results suggested that HMGA2 expression combined with Dox-activated checkpoint control results in more cells undergoing G₂/M accumulation and apoptosis. However, an alternate explanation for these results was that HMGA2 may regulate steady-state topo II levels. Topo IIα is the key enzyme target of Dox, and topo II levels are inversely correlated with chemoresistance to Dox. To determine whether this was the case, steady-state levels of topo II following Dox treatment were measured in HS578T cells in the presence and absence of HMGA2. Topo II levels remained steady over the course of treatment, and were independent of HMGA2. Similar results were obtained using HeLa and HeLa/HMGA2 cells. This eliminated the possibility that HMGA2 governs the cellular response to Dox-mediated genotoxicity by modulating steady-state topo IIα levels.

Genomic DNA is continuously exposed to endogenous and exogenous agents that elicit different forms of DNA damage. Repair of this damage or elimination of cells that have accumulated severe damage is crucial to maintaining genomic integrity and modulating resistance to chemotherapeutic agents. Cellular responses to genotoxic stress range from cell cycle arrest, which allows for DNA repair, to apoptosis when damage is irreparable (Motoyama 2004; Norbury 2004). In addition, cell cycle checkpoint control is an important part of the DNA damage signaling network because it prevents replication of damaged DNA.

DSBs arise endogenously during processes such as meiosis and DNA replication (Riballo 2004). Two major DSB repair pathways are homologous recombination and DNA nonhomologous end-joining (Jeggo 1998; Couedel 2004). DSBs also activate signaling cascades that render cell cycle checkpoint arrest and/or apoptosis (Rouse 2002; Shiloh 2003). It was hypothesized that HMGA2 may augment Dox-elicited growth inhibition by modulating cellular responses to the genotoxic stress of Dox-induced DSBs. This hypothesis was initially tested by analyzing the effect of Dox treatment on ataxia telangiectasia-mutated kinase (ATM) activation. ATM is a member of the phosphatidylinositol 3-kinase (Pi 3-K)-related protein kinase (PIKK) family of enzymes, which have been proposed to contribute to the major signaling pathways underlying surveillance and maintenance of genome integrity (Shiloh 2003; Kurz 2004). ATM plays an integral role in cell cycle checkpoint control, resulting in the activation of either apoptotic or anti-apoptotic signaling pathways (Pearce 2001; Shiloh 2004). Evidence shows that ATM-null mice are susceptible to constant genotoxic stress (Weizman 2003; Ch'ang 2005).

The PIKK family of DNA damage-activated serine/threonine protein kinases also includes DNA-dependent protein kinase (DNA-PK) and ATM- and Rad3-related kinase (ATR) (Durocher 2001). DNA-PK, ATM, and ATR share sequence homology and many of the same substrates, but differ with respect to the types of genotoxic stress that render their activation. ATM primarily responds to agents that cause DNA double-strand breaks (DSBs), whereas ATR signals in response to the agents that cause bulky adducts on DNA or otherwise cause stalling of replication forks and generated of single-strand DNA breaks (Abraham 2001; Rouse 2002). The DNA-PK holoenzyme is a heterodimer of 70- and 80-kDa subunits (Ku70 and Ku80, respectively) that bind to DNA DSBs, recruiting and activating a 470-kDa catalytic subunit (DNA-PKcs), and playing an essential role in the non-homologous end joining (NHEJ) process (Shiloh 2004). The heterodimer of Ku70 and Ku80 acts as the regulatory part of the DNA-PK and initiates the DSB repair process by recruiting DNA-PKcs after binding to DSBs (Featherstone 1999). This complex recruits other proteins including DNA ligase IV and XRCC4, which are necessary for DNA end-joining. Recently, DNA polymerase μ has also been reported to be involved in the DNA-PK signaling pathway (Mahajan 2002). Numerous studies have shown that cells lacking DNA-PK are hypersensitive to ionizing radiation and cross-linking agents, and defective in DSB repair (Burma 2004).

Dox was found to induce ATM Ser 981 phosphorylation in ATM-proficient YZ5 cells. The effect of Dox treatment on cell viability in ATM-deficient pEBS7 cells and ATM-proficient YZ5 cells was measured by MTT assay. Dox decreased cell viability in both cell types, but this decrease was less pronounced in the ATM-negative pEBS7 cells.

Activated ATM, ATR, and DNA-PK can initiate G₂ cell cycle arrest, signaling through Chk1, Chk2, or p53. In addition, they can phosphorylate histone 2A variant X (H2AX). H2AX is deposited randomly throughout chromatin, comprising approximately 10% of total nucleosomal histone H2A (Pilch 2003). A highly conserved serine residue at position 139 of H2AX (Ser 39) is phosphorylated by ATM, ATR, or DNA-PK in response to DNA damage (Rogakou 1998; Burma 2001; Ward 2001). Phosphorylation of H2AX is thought to amplify the DNA damage signal by enhancing and stabilizing the recruitment of DNA damage sensor proteins and DNA repair proteins in response to DNA damage or replication stress (Zimmerman 2004). For example, H2AX phosphorylation is required for recruitment of stable formation of NBS1, 53BP1, MDC1, and BRCA1 complex to damaged sites (Paull 2000; Celeste 2002; Wang 2002; Celeste 2003; Stewart 2003). H2AX Ser 39 phosphorylation is a hallmark of DNA DSBs, with hundreds to thousands of H2AX molecules phosphorylated over several megabases flanking the sites of a single DSB (Rogakou 1998; Rogakou 1999).

The effect of Dox treatment on H2AX phosphorylation was measured in Pa-4, Pa-4/HMGA2, HeLa, and HeLa/HMGA2 cells. Little or no basal H2AX phosphorylation was observed in Pa-4 and HeLa cells, and Dox treatment led to rapid H2AX phosphorylation in both cell types. In Pa-4/HMGA2, HeLa/HMGA2, and HS578T cells, on the other hand, a persistent high level of basal H2AX phosphorylation was observed, resulting in an ablation of Dox-mediated induction of H2AX phosphorylation. Treatment of cells with caffeine, which inhibits ATM, or NU7026, which inhibits DNA-PK, decreased Dox-induced H2AX phosphorylation in Pa-4, HeLa, and HCC1419 cells, but had no effect on basal H2AX phosphorylation levels in Pa-4/HMGA2, HeLa/HMGA2, and HS578T cells. These results suggested that the capacity of both caffeine and NU7026 to repress Dox-elicited H2AX phosphorylation is inversely correlated to HMGA2 expression. Treatment with caffeine decreased the number of Pa-4 and Pa-4/HMGA2 cells in G₂/M following Dox treatment, suggesting that Dox-mediated cell cycle arrest is ATM- and/or ATR-dependent. Treatment with caffeine increased Dox-induced apoptosis in Pa-4/HMGA2 and HeLa/HMGA2 cells, but had no effect on Dox-induced apoptosis in Pa-4 and HeLa cells. These results make it clear that there is a potential combinatory effect between Dox and caffeine to commit more HMGA2-expressing cells to apoptosis.

To determine which PIKK mediates Dox-induced and/or HMGA2-associated basal H2AX phosphorylation, H2AX phosphorylation was measured in the presence and absence of Dox in pEBS7 (ATM−), YZ5 (ATM+), DNA-PKcs-deficient MO59J, and U2OS.GK41 cells. ATM-deficient pEBS7 cells showed high levels of basal H2AX phosphorylation and a lack of Dox-mediated induction similar to what was seen in HMGA2-expressing Pa-4/HMGA2, HeLa/HMGA2, and HS578T cells. On the other hand, ATM-proficient YZ5 cells, DNA-PKcs-deficient MO59J cells, U2OS.GK41 expressing ATR-kd, and U2OS.GK41 cells not expressing ATR-kd all exhibited low levels of basal H2AX phosphorylation and a substantial increase in H2AX phosphorylation following Dox treatment.

Silencing of HMGA2 expression using siRNA treatment virtually blocked H2AX phosphorylation in HS578T cells, and decreased the level of Dox-mediated cell killing in both HeLa/HMGA2 and HS578T cells. This effect was more dramatic in HeLa/HMGA2 cells than in HS578T cells, which may be due to other effects inherent in the siRNA strategy. For example, siRNAs have been demonstrated to induce a global signaling response, including the induction of interferon-mediated JAK/STAT pathway activation (Sledz 2003; Kim 2004; Sledz 2004; Hornung 2005).

DSBs may arise from blocked or collapsed replication forks generated by treatment with cisplatin (De Silva 2000), an intrastrand cross-linker. In addition, DSBs may be induced by X-ray irradiation. Thus, the effect of HMGA2 expression on cell survival following exposure to cisplatin or X-ray radiation was assessed. Cells that expressed HMGA2 were found to be more sensitive to cisplatin-elicited growth inhibition than those that did not express HMGA2. Cells expressing HMGA2 were also found to be more sensitive to radiation.

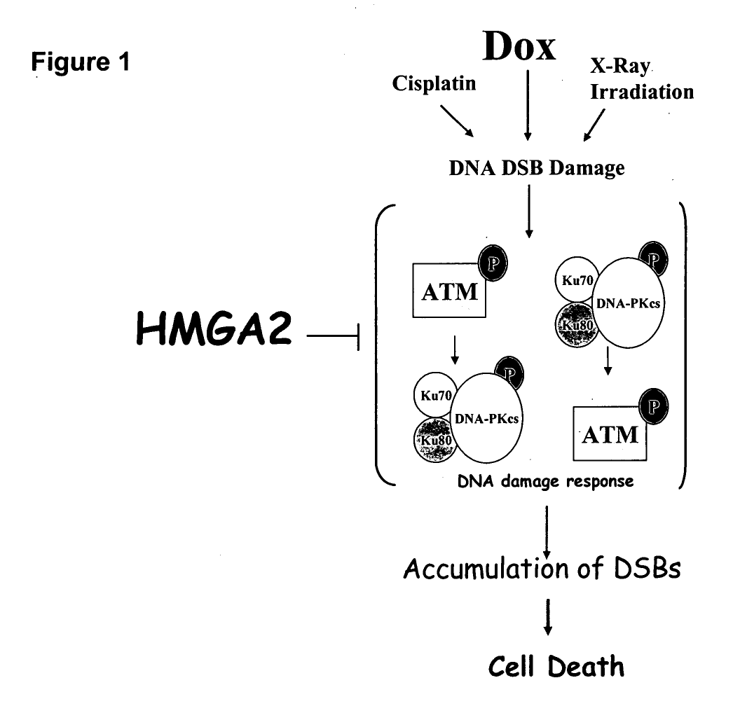

Together, these results indicate that HMGA2 confers persistent basal H2AX phosphorylation in a caffeine- and NU7026-insensitive manner. In addition, HMGA2 expression appears to perturb Dox-elicited DNA damage checkpoint control (i.e., induction of ATM/ATR/DNA-PK-dependent H2AX phosphorylation), which in turn promotes enhanced chemosensitivity towards Dox treatment in HMGA2-expressing cells. The persistent H2AX phosphorylation mediated by HMGA2 may represent a key step that determines ensuing events in the signal transduction pathway in response to subsequent genotoxic stress. Without wishing to be bound by any theory, it is possible that HMGA2 expression results in an adaptive mechanism to consistently phosphorylate H2AX, thus desensitizing H2AX from further phosphorylation by Dox-elicited genotoxicity. By perturbing a signaling pathway underlying basal and DNA damage-dependent PIKK activation that normally functions to maintain genomic integrity, HMGA2 expression increases cytotoxicity following treatment with Dox. A schematic of the role of HMGA2 in enhancing cytotoxicity is set forth in FIG. 1.

In addition to increasing sensitivity to Dox-mediated genotoxicity, HMGA2 expression also increases sensitivity to genotoxicity from treatment with other DNA-damaging therapeutic agents such as cisplatin and X-ray irradiation. Thus, the presence of HMGA2 expression may serve to increase the efficacy of a variety of cytotoxic cancer therapies. This is consistent with previous findings establishing that dysregulation of the cellular DNA damage response may function to promote cancer development (Bartkova 2005; Gorgoulis 2005; Venkitaraman 2005). Along similar lines, it has been suggested that BRCA2-deficient cancer cells can be killed by overloading them with DNA repair inhibitors (Bryant 2005; Farmer 2005). In addition, oncogenic Ras increases sensitivity of colon cancer cells to 5-FU-induced apoptosis (Klampfer 2005).

The enhanced chemosensitivity to treatment with Dox, cisplatin, and X-ray irradiation exhibited by HMGA2-expressing cells suggests that they are more susceptible to the genotoxic stress of DSBs. Since NHEJ can be subdivided into error-free repair and microhomology-mediated error-prone mutagenic repair (Guirouilh-Barbat 2004), experiments were conducted to determine the potential contribution of HMGA2 to NHEJ using an established plasmid end-joining assay (Zhong 2002). The pGL2 plasmid was linearized with HindIII and EcoRI and transfected into HeLa and HeLA/HMGA2 cells, together with a control pRL-TK luciferase plasmid, and cells were treated with Dox. While any end-joining with small deletion or insertion will not affect the luciferase activity detected from the HindIII cut plasmid (cleaved at the linker region between the promoter and coding sequence), only an error-free end-joining can restore the luciferase activity in EcoRI-linearized plasmid (digested at the luciferase coding region). HMGA2 expression was found to decrease both overall and error-free end joining activity, suggesting that HMGA2 inhibits NHEJ generally.

To examine the possibility that there are an increased amount of DNA lesions accumulated in HMGA2-expressing cells prior to Dox treatment, a single cell gel electrophoresis assay in alkaline conditions, or “Comet assay” (Collins 2004), was performed. The Comet assay is used to visualize the extent of endogenous DNA damage based on the principle that denatured, cleaved DNA fragments tend to migrate out of the nucleus when current is applied, whereas undamaged DNA migrates slower and remains within the confines of the nucleus. The Comet assay revealed a two-fold increase in endogenous DNA damage in PA-4 cells expressing HMGA2, confirming the earlier results showing that HMGA induces accumulation of γ-H2AX.

The effect of HMGA2 expression on the sensitivity of HepG2, Hep3B, and CL48 cells was measured using an MTT assay. HepG2 and Hep3B are hepatoma cell lines expressing high levels of endogenous HMGA2, while CL48 is an immortalized liver cell line with low HMGA2 expression levels. As expected, HepG2 and Hep3B cells exhibited greater sensitivity to both Dox and cisplatin than CL48 cells, indicating that the chemosensitizing effects of HMGA2 towards Dox and cisplatin are not limited to one type of cancer cell. To verify that HMGA2 does not increase chemosensitization in general, Taxol-induced cell growth inhibition was measured in HeLa and HeLa/HMGA2 cells. There was no substantial difference in the response to Taxol between HeLa and HeLa/HMGA2 cells, indicating that the observed HMGA2-mediated chemosensitization is restricted to agents that cause DSBs.

The DNA-PKcs activation profile of cells expressing HMGA2 was investigated. Dox treatment has been reported to lead to rapid and transient phosphorylation of DNA-PKcs at Thr-2609 (pT2609) and Ser-2056 (pS2056), respectively (Chen 2005). Western analyses using anti-phospho-DNA-PKcs-T2609/S2056 and anti-DNA-PKcs revealed that HeLa cells expressing HMGA2 exhibited an increase in DNA-PKcs phosphorylation with no increase in steady-state DNA-PKcs levels following treatment with Dox. These results were recapitulated in HMGA2-expressing Hs578T cells. siRNA assays confirmed that HMGA2 HMGA2 expression is at least partially responsible for sustained DNA-PKcs pT2609 and pS2056 signals following Dox exposure. Quantitative fluorescent immunostaining studies further revealed that HMGA2 expression was accompanied by an increase in the average number and volume of discrete nuclear pT2609-DNA-PKcs foci following Dox treatment. Taken together, these results confirm that HMGA2 augments Dox-induced DNA-PKcs phosphorylation.

Since HMGA2 is a SUMOylation target (Cao, X. and Ann, D.K., unpublished observation), experiments were performed to determine whether SUMOylation is involved in HMGA2-mediated chemosensitization. An HMGA2 mutant was generated containing Lysine to Arginine (Lys→Arg) point mutations at residues 66 and 67, corresponding to the SUMOylation site. Expression of the SUMOylation-defective HMGA2 partially reversed Dox- and cisplatin-induced cytotoxicity, suggesting that SUMOylation may play a role in mediating chemosensitization towards DSBs elicited by HMGA2. Western analyses and quantitative fluorescent immunostaining experiments showed comparable Dox accumulation in the nucleus of HeLa, HeLa/HMGA2, and SUMOylation-defective HeLa/HMGA2(2K/R) cells, leading to the conclusion that HMGA2-mediated chemosensitization to Dox is not due to increased nuclear Dox accumulation.

MTT assays were conducted using Ku70-deficient (Ku70⁻/⁻) MEF cells and their wild-type counterparts and DNA-PKcs-deficient MO59J cells and DNA-PKcs-proficient MO59K cells to confirm the involvement of DNA-PK in mediating HMGA2 chemosensitization to DNA damage. Ku70⁻/⁻ cells showed a marked decrease in viability following Dox or cisplatin treatment versus their wild-type counterparts, while MO59J cells exhibited a similar increase versus MO59K cells following Dox treatment. Given that both pT2609 and pS2056 signals of DNA-PKcs are associated with DSB-induced genotoxicity, and that both Ku70 and DNA-PKcs are required for the proper assembly and function of the DNA-PK complex following Dox exposure, it appears that HMGA2 confers cellular sensitivity towards Dox by interfering with DNA-PK-mediated DSB repair.

Co-immunoprecipitation assays were conducted to determine whether HMGA2 interacts directly with either subunit of the DNA-PK complex to dysregulate DNA-PK activation. Ku80 co-immunoprecipitated with Ku70, and HMGA2 was detected in the immunocomplexes of Ku80/Ku70. These results were confirmed by in vitro pull-down experiments, which showed that individually translated Ku80 was able to be pulled down by HMGA2. This cell-free experimental system confirmed that the interaction between Ku80 and HMGA2 occurs at least in part via a direct protein-protein interaction in vitro.

To determine whether Dox treatment affects the protein-protein interaction between HMGA2 and Ku80/Ku70, HeLa and HeLa/HMGA2 cells were treated with Dox and then subjected to immunoprecipitation assays using anti-HA tag or anti-Ku80 antibody. Dox treatment increased the amount of DNA-PKcs detected in Ku80 immunocomplexes prepared from cells expressing HMGA2, supporting the notion that HMGA2 potentiates Dox-induced DSBs as more DNA-PKcs being recruited to the sites of Ku80/Ku70-bound DSBs. However, Dox-treatment resulted in a decrease in anti-Ku80-immunoprecipitated HMGA2 without affecting the steady-state level of Ku70 detected in the Ku80-immunocomplexes in HeLa/HMGA2 cells. The effect of Dox treatment on the interaction between HMGA2 and the Ku70/Ku80 heterodimer was further confirmed by a reciprocal co-immunoprecipitation assay using an anti-HA antibody. DNA-PKcs were detected in the immunocomplexes of HMGA2 prior to Dox treatment in HeLa/HMGA2 cells, supporting the idea that HMGA2 promotes an accumulation of endogenous DNA DSBs. To verify that the interaction of HMGA2 and Ku80/Ku70 was not an artifact, the interaction between endogenous HMGA2 and Ku80/Ku70 in Hs578T breast cancer cells was examined. Endogenous HMGA2 interacted with Ku80/Ku70, and this interaction was disrupted upon Dox-treatment. Overall, these results suggest that HMGA2 dysregulates the DNA damage response by forming complexes with Ku80/Ku70 and/or DNA-PKcs prior to Dox treatment, and dissociates from such complexes following Dox exposure.

To examine the biological consequence of HMGA2 association with Ku80/Ku70, experiments were conducted to determine the regions of HMGA2 that are required to form a complex with Ku80. Since the three DNA-binding AT-hook domains and both the N- and C-termini of HMGA2 have been shown to have different roles in cell proliferation, transcription regulation, and protein-protein interactions (Noro 2003), three truncated forms of HMGA2 were created carrying deletions of either the N-terminus (Δ1-24), C-terminus (Δ83-109) or C-terminus with the AT-hook III (Δ66-109). These constructs were transiently transfected into HeLa cells, and co-immunoprecipitation studies were performed to analyze the ability of each to interact with Ku80. HMGA2(Δ1-24) exhibited the same interaction level with Ku80 as wild-type HMGA2, but HMGA2(Δ66-109) and HMGA2(Δ83-109) exhibited more than a 70% decrease and more than a 50% decrease, respectively.

Another HMGA2 (HMGA2(4P/A)) was generated to determine whether the DNA binding ability of the AT hooks is necessary for the HMGA2/Ku80 interaction. HMGA2(4P/A) contained four Pro→Ala mutations at residues located in the second and third AT-binding hooks. Mutation of these residues has been reported to abolish the ability of HMGA2 to bind DNA (Himes 2000). HMGA2(4P/A exhibited almost no Ku80 binding ability, indicating that the interaction between HMGA2 and Ku80 is reliant at least in part on the DNA binding ability of HMGA2.

Additional mutant constructs were generated to more precisely map the DNA binding region of HMGA2 involved in HMGA2/Ku80 complex formation. These mutants, HMGA2(2P/A) and HMGA2(3P/A), contained Pro→Ala mutations at positions 48/52 (AT-hook II) and 48/52/80 (AT-hook II and III), respectively. The Pro residues in the second AT-hook were found to be essential to the HMGA2/Ku80 interaction. Thus, HMGA2 appears to complex with Ku80 via both protein-protein and protein-DNA interactions.

To examine the relevance of HMGA2 complex formation with Ku80/Ku70, cell viability was measured in Dox-treated HeLa cells transiently transfected with HMGA2 or mutant HMGA2 constructs. As expected, transient transfection with HMGA2 increased Dox sensitivity. Likewise, transient transfection with HMGA2(Δ1-24), which retained the ability to interact with Ku80/Ku70, increased Dox sensitivity. By contrast, cells transiently transfected with HMGA2(Δ83-109) or HMGA2(4P/A) exhibited greater resistance to Dox treatment. This increased Dox resistance may be due to protein-protein interactions arising from the remaining portion of HMGA2. Taken together, these results suggest that the ability of HMGA2 to complex with Ku80 contributes, at least in part, to the HMGA2-mediated Dox chemosensitization.

Based on the reciprocal dependency between DNA-PK and ATM (Chan 2002; Peng 2005), experiments were performed to determine whether the sustained DNA-PKcs phosphorylation profile is accompanied with the dysregulated DNA damage-triggered signaling of other PIKK members. The ATM activation profile of HeLa cells and HeLa/HMGA2 cells in response to Dox treatment was probed, and it was found that HMGA2 delayed ATM Ser-1981 activation following Dox exposure.

Both DNA-PK and ATM have been shown to initiate DNA damage signaling by enhancing phosphorylation of p53 at Ser-15 and Chk2 at Thr-68 (Wang 2000; Lukas 2003). To determine the role of DNA-PK in Dox-induced p53 and Chk2 phosphorylation, the phosphorylation profiles of p53 and Chk2 were examined in MO59J (DNA-PKcs-deficient) and MO59K (DNA-PKcs-proficient) cells treated with Dox. Dox treatment resulted in robust p53 phosphorylation at Ser-15 (pSer15-p53) in both cell types, but Chk2 phosphorylation at Thr-68 (pThr68-Chk2) was barely detectable in MO59J cells. Together, these results suggest that proper DNA-PKcs function is required for Dox-induced pSer1981-ATM and pThr68-Chk2 signal, but not pSer15-p53 signal

pSer15-p53 and pThr68-Chk2 profiles following Dox exposure was examined. Exposure of HeLa cells to Dox caused induction of pSer15-p53 and pThr68-Chk2 signals. There was a noticeable accumulation of pSer15-p53 and attenuation of pThr68-Chk2 following Dox treatment in HeLa/HMGA2 cells as compared to HeLa and HeLa/HMGA2(2K/R) cells. Thus, HMGA2 appears to disrupt the DNA damage response by interfering with the activation of DNA-PKcs, ATM, and Chk2.

Both DNA-PKcs and ATM are involved in checkpoint control and apoptosis through the activation of downstream Chk2 and p53 following genotoxic stress (Geske 2000; Motoyama 2004). Since the results discussed above indicate that HMGA2 dysregulates DNA-PK/ATM activation and that HMGA2-expressing cells exhibit a robust pSer15-p53 but an attenuated pThr68-Chk2 signal following Dox treatment, the involvement of other pro-cell death molecules (e.g., caspase-3, PUMA, and NOXA) in HMGA2-promoted chemosensitization was examined by Western analyses. Caspase-3 cleavage fragment levels, a hallmark of caspase-3 activation, were increased in HeLa/HMGA2, HepG2 and Hs578T cells following Dox exposure. Dox treatment increased steady-state p53 levels in HeLa, HeLa/HMGA2, CL48, and HepG2 cells.

The up-regulation or activation of pro-apoptotic BH3-only proteins serves to link cellular stress to the initiation of cell death response (Cory 2003). Expression of the BH3-only proteins PUMA and NOXA is reported to be transcriptionally simulated by p53 following DNA damage (Villunger 2003; Yakovlev 2004; Chipuk 2005). To determine the effect of HMGA2 expression on PUMA and NOXA transcriptional activation, quantitative real-time RT-PCR was performed using lysates from various Dox-treated cell types. Dox induced marked transcriptional activation of PUMA and NOXA in CL48 and HepG2 cells, but there was minimal induction in Hep3B cells and the various HeLa cell types. Thus, Dox treatment induces PUMA and NOXA expression in PUMA and NOXA cells, but PUMA and NOXA expression does not correlate with the decreased viability and enhanced activation of caspase-3 observed in HMGA2-expressing cells following Dox treatment.

Taken together, the experimental results set forth herein show that dysregulation of the DSB damage signaling pathway by HMGA2 is one of the major mechanisms underlying enhancement of cell growth inhibition following Dox exposure. Thus, HMGA2-promoted chemosensitization can be used alone or as a complement to p53 in conferring Dox- or other DSBs-triggered cytotoxicity.

“DNA-damaging therapeutic agent” refers to any agent or treatment method that induces DNA damage when applied to a cell. Examples of DNA-damaging therapeutic agents include Dox, cisplatin, adriamycin, etoposide (VP-16), verapamil, podophyllotoxin, 5-fluorouracil (5-FU), camptothecin, actinomycin-D, mitomycin C, X-ray irradiation, gamma irradiation, UV irradiation, and microwaves. DNA-damaging therapeutic agents are mainstays of cancer therapy, and have achieved impressive clinical results. However, the usage of these agents in treating cancer patients is often plagued by side effects such as cardiotoxicity, congestive heart failure, and myelosuppression, cisplatin-related nephrotoxicity, emetogenesis, and dose-limiting neurotoxicity (Minotti 2004; Wang, D. 2005). Side effects related to Dox administration have generally limited the maximum cumulative dosage of Dox in a subject to 450-550 mg. To alleviate Dox-related toxicity, it has often been necessary to decrease administration dosages, which can compromise the effectiveness of treatment. In addition, cancer patients are often treated with a combination of multiple chemotherapeutic agents. For example, breast cancer patients are often treated with a combination of taxane and anthracycline. This use of multiple agents may increase toxicity risk. The results disclosed herein establish that in addition to promoting tumorigenesis, HMGA2 expression also serves as a useful therapeutic indicator for identifying subjects that are likely to respond favorably to treatment with a DNA-damaging therapeutic agent such as Dox, cisplatin, or X-ray irradiation. By selectively utilizing these therapies in patients with cancer cells expressing HMGA2, the efficacy of these therapies may be increased and the number of patients developing negative side effects may be decreased. The feasibility of such an approach is underscored by recent studies supporting the value of individualized pharmacotherapy (Watters 2003; Ross 2004). Utilization of a pharmacogenetic approach is likely to decrease the number of agents that must be administered to a subject, decreasing the risk of toxicity.

In certain embodiments, methods are provided for increasing chemosensitivity of a cancer cell to treatment with a therapeutic agent related to a DNA damage pathway by introducing into the cancer cell a polynucleotide encoding an HMGA2 polypeptide or a fragment thereof. Introduction of a polynucleotide “increases chemosensitivity” of a cancer cell if it increases the likelihood that the cell will be killed or rendered unviable and/or increases the rate at which the cell is killed or rendered unviable following treatment with a DNA-damaging agent. Introduction of the polynucleotide may be carried out in any manner that allows for expression of the polynucleotide following introduction. For example, the polynucleotide may be introduced into the cancer cell by a transfection method such as for example DEAE-Dextran-mediated, calcium-phosphate-mediated, or cationic lipid-mediated transfection, electroporation, nucleofection, lipofection, microinjection, ballistic introduction, or scrape loading. In certain embodiments, the polynucleotide may be introduced into the cancer cell using a viral-based delivery system, such as for example a retroviral, adenoviral, or cytomegalovirus vector system. Polynucleotides encoding HMGA2 or a fragment thereof may be introduced into multiple cancer cells from a single subject.

In certain embodiments, methods are provided for increasing the effectiveness of treatment with a DNA-damaging therapeutic agent such as Dox, cisplatin, or X-ray irradiation in a subject with cancer by administering to the subject a therapeutically effective amount of an HMGA2 polypeptide or fragment thereof. In these embodiments, the polypeptide or fragment thereof may be administered by any pathway known in the art that results in the polypeptide or fragment thereof contacting one or more cancer cells. For example, the polypeptide or fragment thereof may be delivered to a subject via intratumoral injection, or it may be administered systemically in conjunction with a targeting agent that directs the polypeptide to a cancer cell. The phrase “fragment thereof” as used herein with regards to HMGA2 refers to a fragment of HMGA2 that retains the ability to increase chemosensitivity of a cell or group of cells to Dox, cisplatin, or radiation treatment. An HMGA2 polypeptide or fragment thereof “increases the effectiveness” of a DNA-damaging therapeutic agent if it increases the ability of the agent to kill or render non-viable a cancer cell or group of cells and/or increases the rate at which the agent is able to kill or render non-viable a cancer cell or group of cells.

In certain embodiments, methods are provided for determining the likelihood that treatment of cancer with a DNA-damaging agent such as Dox, cisplatin, or X-ray irradiation will be effective, or for determining the extent of this effectiveness, by measuring HMGA2 expression levels in one or more cancer cells. In addition, kits are provided for carrying out this measurement. “HMGA2 expression levels” may refer protein or mRNA levels, and may be measured by a variety of means well known in the art. For example, HMGA2 mRNA levels may be measured using a PCR-based approach, using one or more sets of primers specific to HMGA2, while HMGA2 polypeptide levels may be measured by Western analysis, using one or more antibodies specific to HMGA2 or fragments thereof.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### HMGA2 Expression Increases Dox-Mediated Growth Inhibition

Pa-4 and Pa-4/HMGA2 Cells

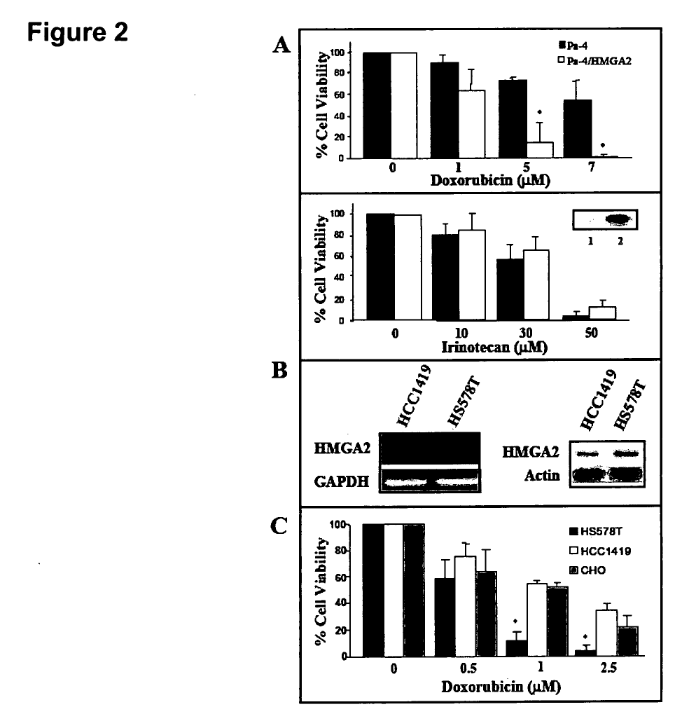

Salivary Pa-4 and Pa-4/HMGA2 cells were grown in DMEM/F12-based medium supplemented with 2% fetal bovine serum (FBS) and incubated at 35° C. as described previously (Li 1997). HMGA2 expression in each cell type was determined by Western analysis. Cells were lysed in boiling SDS loading buffer, heated for 10 minutes, and centrifuged at 13,000 rpm for 10 minutes. Supernatants were collected and protein concentrations were determined by Bradford assay. 80 μg of proteins were loaded onto SDS-PAGE gels, and the separated proteins were transferred to IMMOBILON membranes (Millipore, Billerica, Mass.) overnight at 4° C. Membranes were probed with polyclonal mono-specific chicken anti-HMGA2 antibody against the peptide sequence MSARGEGAGQPSTSA (SEQ. ID. NO: 1) (Aves Labs, Tigard, Oreg.), which is located at the amino-terminus of HMGA2. Western analyses confirmed that HMGA2 expression levels were high in Pa-4/HMGA2 cells, and low or non-existent in Pa-4 cells (FIG. 2A, lower panel, inset).

Cells were seeded into 24-well plates to obtain a confluency of 35-50%, at which point they were treated with varying concentrations of Dox (0-7 μM) or irinotecan (0-50 μM). After treatment, the cell medium was changed daily for three days. 72 hours after treatment, 0.2 ml of 0.1 mg/ml 3-[4,5-dimethylthiazol-2-yl]-2,5-diphenyltetrazolium bromide (MTT) (Sigma, St. Louis, Mo.) in OptiMEM I (Invitrogen, Carlsbad, Calif.) was added to each well, and plates were incubated at 37° C. for 1.5 hours. The MTT solution was then aspirated and 0.2 ml isopropanol was added to each well to dissolve the formazan crystals. Absorbance was read immediately at 540 nm in a scanning multiwell spectrophotometer.

Dox treatment decreased cell viability in a dose-dependent manner in both Pa-4 and Pa-4/HMGA2 cells (FIG. 2A, upper panel). However, this decrease was much larger in Pa-4/HMGA2 cells than it was in Pa-4 cells, suggesting that HMGA2 enhances Dox-induced cytotoxicity. Treatment with irinotecan also resulted in dose-dependent growth inhibition in both cell types, with little difference observed between the two cell types (FIG. 2A, lower panel).

HeLa and HeLa/HMGA2 Cells

HeLa/HMGA2 cells were generated by transfecting parental HeLa cells with pcDNA3.1/HA-HMGA2 vector using Lipofectamine 2000 transfection reagent (Invitrogen, CA). Cells were cultured in DMEM medium supplemented with 1 mg/ml G418 (Invitrogen, CA). After two weeks, single-cell clones were selected and further cultured in DMEM medium supplemented with 0.5 mg/ml G418 for four weeks. HMGA2 expression was confirmed by Western analysis using an anti-HA antibody (Chemicon, N.J.). Cell lines expressing the highest level of HMGA2 were selected for further study, and were maintained in DMEM supplemented with 10% FBS and 1% streptomycin/penicillin.

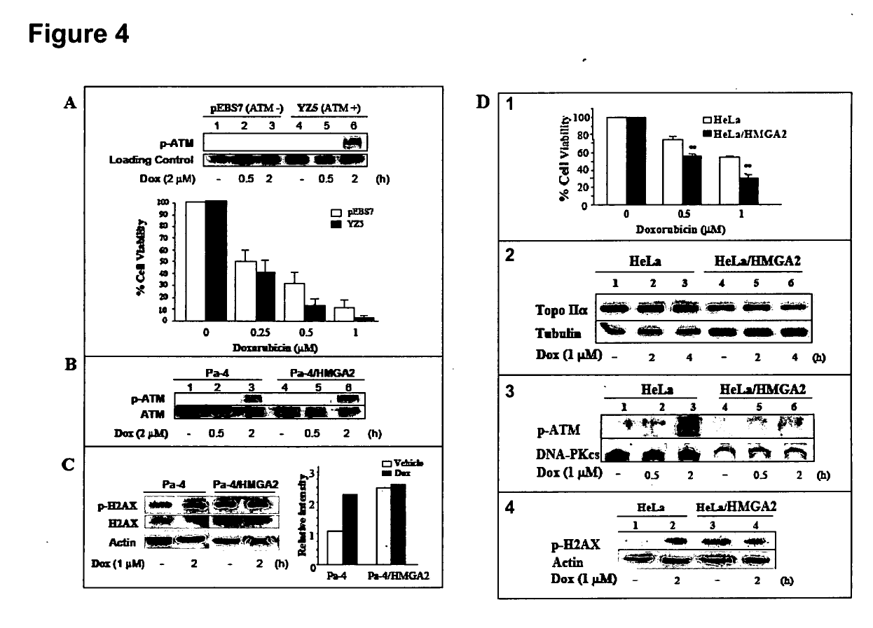

HeLa and HeLa/HMGA2 cells were treated with varying concentrations of Dox (0-1 μM), and cell viability was measured by MTT assay as described above. Results were similar to those observed in Pa-4 and Pa-4/HMGA2 cells, with HMGA2 expression enhancing the chemosensitivity of cells to Dox (FIG. 4D, panel 1). HS578T, HCC1419, and CHO cells

Breast cancer HS578T cells were grown in DMEM plus 10% FBS, supplemented with 10 μg/ml insulin. Breast cancer HCC1419 cells were grown in RPMI supplemented with 4.5 g/L glucose, 10 mM HEPES, and 1 mM sodium pyruvate. CHO cells were maintained in MEM with alpha-modification (Sigma). All cells were cultured in a humidified incubator at 37° C. with 5% CO₂. The HS578T and HCC1419 cells were both estrogen receptor (ER)— and progesterone receptor (PR)-negative.

CHO cells had previously been shown to be deficient in endogenous HMGA2 expression. Endogenous HMGA2 levels were measured in HS578T and HCC1419 cells by RT-PCR using equal amounts of total RNA from each cell type. GADPH levels were also measured as a control to validate the RT-PCR reaction and quantitation of RNAs. RNA was extracted using Trizol reagent (Invitrogen) followed by DNA-free DNase treatment (Ambion). cDNA synthesis and PCR reactions were carried out using ThermoScript RT-PCR System (Invitrogen). The primer pairs for each PCR reaction are set forth in SEQ. ID. NOs:2-5.

These results indicated that steady-state HMGA2 expression levels were higher in HS578T cells than in HCC1419 cells (FIG. 2B, left panel). The results were confirmed by Western analyses using antibodies against HMGA2 (FIG. 2B, right panel). Control Western analyses were performed using monoclonal anti-actin antibodies (Chemicon, Temecula, Calif.).

Cell viability in the presence of varying concentrations of Dox was measured by MTT assay as described above. Dox was administered at concentrations ranging from 0 to 2.5 μM, and cell viability was measured at 72 hours after treatment. Dox decreased cell viability in a dose-dependent manner in all three cell types (FIG. 2C). However, HS578T cells were substantially more sensitive to Dox-mediated growth inhibition than the other two cell types. This supports the idea that HMGA2 expression sensitizes the cellular response to Dox-induced genotoxicity.

### Example 2

### HMGA2 expression increases Dox-mediated G₂/M arrest

Pa-4 and Pa-4/HMGA2 cells were seeded at 50-80% confluency in 35 mm dishes and serum starved overnight to synchronize cell cycle. Synchronized cells were treated with 1 μM Dox or vehicle for 24 hours, then fixed in 70% ethanol overnight and stained with 20 μg/ml propidium iodide. The percentage of cells in G₁ versus G₂/M was then determined by FACS analysis at the Norris Cancer Center Flow Cytometry Core Facility using FACSCaliber (Becton Dickinson, Franklin Lakes, N.J.).

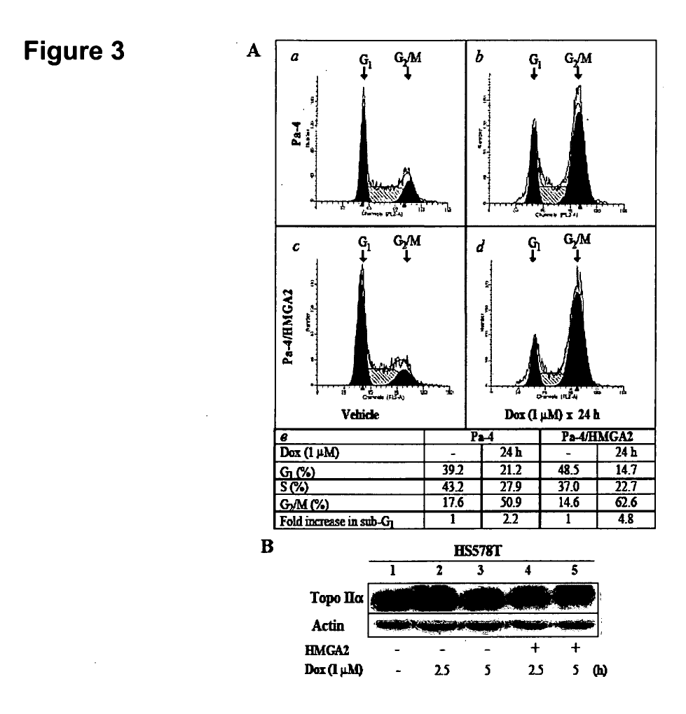

When treated with vehicle only, 17.6% of Pa-4 cells and 14.6% of Pa-4/HMGA2 cells accumulated in G₂/M phase (FIG. 3A, panels a and c). When treated with Dox, however, the percentage of cells in G₂/M phase increased to 50.9% of Pa-4 cells and 62.6% of Pa-4/HMGA2 (FIG. 3A, panels b and d). The percentage of cells in each phase of the cell cycle is summarized in FIG. 3A, panel e). These results were expected since Dox is known to delay the G₂/M transition (Mikhailov 2004).

### Example 3

### HMGA2 Expression Increases Dox-Mediated Apoptosis

Apoptosis of Pa-4 and Pa-4/HMGA2 cells was quantified by assessing the sub-G, cell population, a hallmark of cell apoptosis. The percentage of G₁ Pa-4/HMGA2 cells was increased five-fold 24 hours after Dox treatment, versus an increase of only two-fold in Pa-4 cells (FIG. 3A, panel e).

### Example 4

### HMGA2 Expression has no Effect on Steady-State Topo II Levels in Dox Treated Cells

HS578T cells were treated with 1 μM Dox, and at various time points cell lysates were removed and subjected to Western analyses using monoclonal anti-topo IIα antibody (BD Biosciences, San Jose, Calif.). Control Western analyses were performed using monoclonal anti-actin antibodies. Steady-state topo II levels remained comparable over the time course of Dox treatment, and were independent of HMGA2 expression (FIG. 3B).

The experiment was repeated using HeLa and HeLa/HMGA2 cells. As with the HS578T cells, HMGA2 had no effect on steady-state topo II levels (FIG. 4D, panel 2).

### Example 5

### Dox Treatment Induces ATM Phosphorylation

ATM-deficient pEBS7 cells and ATM-proficient YZ5 cells were maintained in DMEM and 10% FBS with 100 μg/ml hygromycin at 37° C. with 5% CO₂. pEBS7 and YZ5 are stable clones derived from ATM-deficient fibroblasts (AT22IJE-T) with empty vector and Flag-tagged wild-type ATM, respectively (Li 2000). Cells were treated with 2 μM Dox or vehicle only. At various time points after treatment, equal amounts of protein lysates were removed and subjected to Western analyses using monoclonal anti-phospho-ATM antibodies (Ser1981) (Upstate Cell Signaling, Waltham, Mass.). ATM Ser1981 phosphorylation was detected between 0.5 and 2 hours after Dox treatment in ATM-proficient YZ5 cells (FIG. 4A, upper panel, lanes 5 and 6), indicating that Dox treatment activates the ATM cascade.

Viability of ATM (−/−) pEBS7 and ATM-stably transfected YZ5 cells in the presence of varying concentrations of Dox was measured using the MTT assay protocol described in Example 1. Dox was administered at concentrations ranging from 0-1 μM, and cell viability was measured at 72 hours after treatment. Dox treatment decreased cell viability in both cell types in a dose-dependent manner, but this decrease was less pronounced in the ATM-negative pEBS7 cells (FIG. 4A, lower panel).

### Example 6

### HMGA2 Expression is Associated with High Levels of Basal H2AX Phosphorylation and Ablation of Dox-Induced H2AX Phosphorylation

Pa-4 and Pa-4/HMGA2 Cells

To ascertain whether HMGA2 effects Dox-induced ATM signaling activation, phosphorylation of ATM Ser1981 and H2AX Ser139 was assayed in the presence and absence of Dox (2 μM in ATM assays, 1 μM in H2AX assays). At various timepoints after Dox treatment, cell lysates were removed and subjected to Western analyses using monoclonal anti-phospho-ATM antibodies (Ser1981), polyclonal anti-ATM antibodies (Santa Cruz Biotechnology, Santa Cruz, Calif.), monoclonal anti-phospho-H2AX antibodies (Ser 39) (Upstate Cell Signaling), or anti-H2AX antibodies. Control Western analyses were performed using monoclonal anti-actin antibodies. As expected, treatment with Dox led to rapid phosphorylation of ATM (FIG. 4B) and
H2AX (FIG. 4C) in Pa-4 cells. Dox-mediated phosphorylation of ATM was roughly equal in Pa-4 and Pa-4/HMGA2 cells (FIG. 4B). Interestingly, a persistent high level of basal H2AX phosphorylation was observed in Pa-4/HMGA2 cells (FIG. 4C). This resulted in a subsequent ablation of Dox-mediated induction of H2AX phosphorylation.
   HeLa and HeLa/HMGA2 cells

The effect of HMGA2 on ATM and H2AX phosphorylation in HeLa cells was measured using the protocol described above. Dox treatment led to rapid phosphorylation of ATM in HeLa cells, but interestingly no ATM phosphorylation was observed in HeLa/HMGA2 cells. As for H2AX, results were similar to those observed in Pa-4 cells. A high level of basal H2AX phosphorylation was observed in HeLa/HMGA2 cells, resulting in an ablation of Dox-mediated induction of phosphorylation.

### Example 7

### High Levels of Basal H2AX Phosphorylation Associated with HMGA2 Expression are Caffeine- and NU7026-Insensitive

To determine whether the observed differences in Dox-induced H2AX phosphorylation in the presence versus the absence of HMGA2 were mediated by ATM or other related protein kinases, such as ATR or DNA-PK, caffeine (Hall-Jackson 1999; Sarkaria 1999; Zhou 2000) and NU7026 were used to uncouple these kinases. Caffeine has been reported to inhibit ATM and ATR at an IC₅₀ of 0.2 mM and 1.1 mM, respectively (Cortez 2003). NU7026 is a novel DNA-PKcs inhibitor that is highly selective for DNA-PK, but inactive against ATM and ATR (Veuger 2003; Willmore 2004).

Pa-4 and Pa-4/HMGA2 Cells

Caffeine (Sigma-Aldrich, St. Louis, Mo.) was made into a 50 mM working solution in OptiMEM-I (Invitrogen). NU7026 (Calbiochem, San Diego, Calif.) was dissolved in DMSO at 5 mM and stored at −20° C. Pa-4 and Pa-4/HMGA2 cells were pre-treated with 4 mM caffeine, 10 μM NU7026, or vehicle only for 1 hour, then treated with a combination of 1 μM Dox, 4 mM caffeine, and 10 μM NU7026 for 2 hours. Cell lysates were removed and subjected to Western analyses using monoclonal anti-phospho-H2AX (Ser 139) antibodies. Control Western analyses were performed using monoclonal anti-actin antibodies.

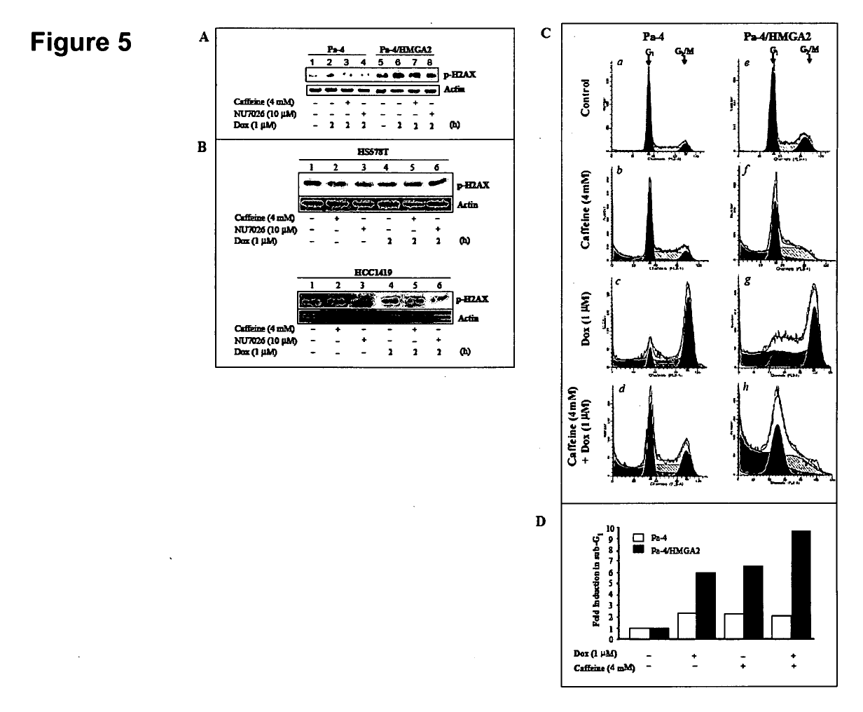

Dox-induced H2AX phosphorylation in Pa-4 cells was attenuated by pre-treatment with caffeine (FIG. 5A, compare lane 2 versus lane 3) and NU7026 (FIG. 5A, compare lane 4 versus lane 2). By contrast, pre-treatment with caffeine or NU7026 had little or no effect on basal H2AX phosphorylation (data not shown) or Dox-induced H2AX phosphorylation in Pa-4/HMGA2 cells (FIG. 5A, compare lanes 7 and 8 versus lane 6).

HS578T and HCC1419 Cells

Experiments were repeated using HS578T and HCC1419 cells treated with 2 μM rather than 1 μM Dox. Dox treatment induced H2AX phosphorylation in HMGA2-deficient HCC1419 cells (FIG. 5B, lower panel, compare lane 1 versus lane 4), but this induction was downregulated in cells that were pre-treated with caffeine or NU7026 (FIG. 5B, lower panel, compare lanes 5 and 6 versus lane 4). In HMGA2-proficient and Dox-sensitive HS578T cells, on the other hand, a significant level of basal H2AX phosphorylation was observed, and there was no increase in H2AX phosphorylation following Dox treatment (FIG. 5B, upper panel, lane 4 versus lane 1). Pre-treatment of HS578T cells with caffeine or NU7026 did not decrease H2AX phosphorylation in either the presence or absence of Dox treatment (FIG. 5B, upper panel, compares lanes 5 and 6 versus lane 4 and lanes 2 and 3 versus lane 1).

### Example 8

### Dox-Induced G₂/M Delay is ATM/ATR Dependent

Pa-4 and Pa-4/HMGA2 cells were seeded at 50-80% confluency in 35 mm dishes and serum starved overnight to synchronize cell cycle. Synchronized cells were pre-treated with 4 mM caffeine or vehicle only for 1 hour, then treated with a combination of 1 μM Dox and 4 mM caffeine for 48 hours. Following treatment, cells were fixed in 70% ethanol overnight, stained with 20 μg/ml propidium iodide, and subjected to FACS analysis. Treatment with caffeine alone had no effect on cell cycle distribution in Pa-4 cells (FIG. 5C, compare panel b versus panel a), but decreased the number of cells in G₂/M in Pa-4/HMGA2 cells (FIG. 5C, compare panel f versus panel e). As expected, treatment with Dox greatly increased the number of cells in G₂/M phase in both Pa-4 (FIG. 5C, compare panel c versus panel a) and Pa-4/HMGA2 (FIG. 5C, compare panel g versus panel a) cells. Treatment with caffeine, however, greatly reduced Dox-induced G₂/M arrest in both cell types (FIG. 5C, compare panel d versus panel c and panel h versus panel g).

Apoptosis was quantified by assessing sub-G₁ cell populations. Treatment with Dox resulted in an increase in apoptosis in both Pa-4 and Pa-4/HMGA2 cells, with a substantially larger increase in Pa-4/HMGA2 cells (FIG. 5D). Similar results were observed when cells were treated with caffeine alone (FIG. 5D). In Pa-4/HMGA2 cells, treatment with both caffeine and Dox resulted in apoptosis levels greater than those observed after treatment with caffeine or Dox alone (FIG. 5D). In Pa-4 cells, however, treatment with caffeine did not increase the rate of Dox-induced apoptosis (FIG. 5D). Similar results were obtained when these experiments were repeated in HeLa and HeLa/HMGA2 cells.

### Example 9

### Identification of PIKK Responsible for H2AX Phosphorylation

To determine which PIKK mediates Dox-induced and/or HMGA2-associated basal H2AX phosphorylation, several cell lines were utilized. These included pEBS7 (ATM−), YZ5 (ATM+), DNA-PKcs-deficient MO59J (Lees-Miller 1995), and U2OS.GK41 cells. MO59J cells were maintained in DMEM plus 10% FBS, in a humidified incubator at 37° C. with 5% CO₂. U2OS.GK41 cells, which harbor doxycycline-inducible ATR-kd (Nghiem 2002), were cultured in DMEM plus 10% FBS supplemented with 400 μg/ml G418 plus 50 μg/ml hygromycin.

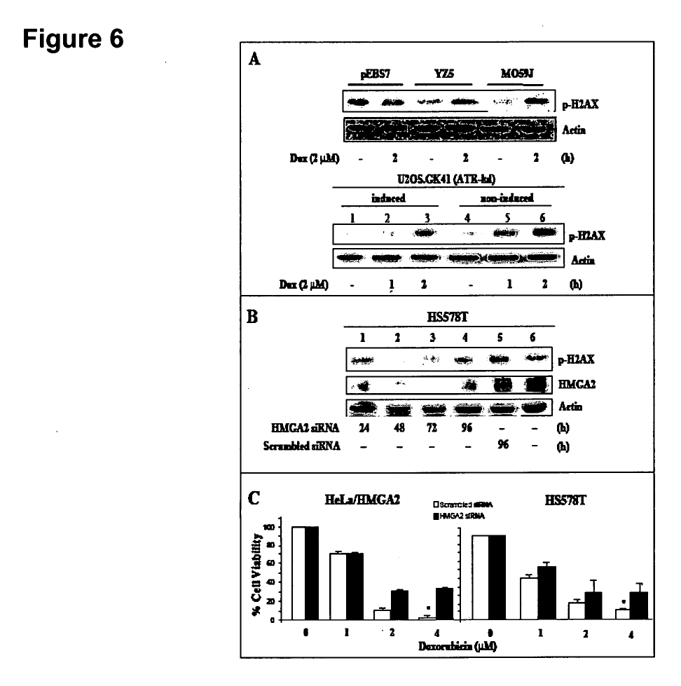

pEBS7, YZ5, and MO59J cells were treated with 2 μM Dox for 2 hours, at which point cell lysates were removed and subjected to Western analyses using anti-phospho-H2AX antibodies. Control Western analyses were performed using monoclonal anti-actin antibodies. ATM-deficient pEBS7 cells displayed a high level of basal H2AX phosphorylation and a lack of Dox-mediated induction (FIG. 6A, upper panel). These results were consistent with those obtained using HMGA2-expressing Pa-4/HMGA2 (FIG. 4C, left panel), HeLa/HMGA2 (FIG. 4D, panel 4), and HS578T (FIG. 5B, upper panel) cells. On the other hand, ATM-proficient YZ5 cells and DNA-PKcs-deficient MO59J cells exhibited low levels of basal H2AX phosphorylation and a substantial increase in H2AX phosphorylation following Dox treatment (FIG. 6A, upper panel).

For experiments using U2OS.GK41 cells, cells were pre-treated with 1 μg/ml doxycycline for 48 prior to induce expression of ATR-kd. Non-induced control cells were pre-treated with vehicle only. Induced and non-induced cells were then treated with 2 μM Dox. At 1 hour and 2 hours after Dox treatment, cell lysates were removed and subjected to Western analyses using anti-phospho-H2AX or anti-actin antibodies. Cells expressing ATR-kd exhibited a delay in Dox-mediated induction of H2AX phosphorylation, and lower phosphorylation levels at 2 hours after Dox treatment (FIG. 6A, lower panel). Neither induced nor non-induced cells exhibited substantial levels of basal H2AX phosphorylation.

### Example 10

### Silencing of HMGA2 Expression Blocks Basal H2AX Phosphorylation

HMGA2 expression was downregulated in HS578T cells using an RNA interference (RNAi) approach. siRNA has been shown previously to be a useful tool for manipulating gene expression or determining gene function (Hannon 2002). Cells were seeded at 90% confluency, and transfected with siRNA using GeneEraser (Stratagene). Cells were transfected with 100 nM HMGA2 siRNA, 100 nM scrambled siRNA, or vehicle only. The oligonucleotides encoding HMGA2 siRNA were 5′-CAGCMTCTGTCGCTAAGGdTdT-3′ (SEQ. ID. NO:6) and 5′-CCTTAGCGACAGATTGCTGdTdT-3′ (SEQ. ID. NO:7), and the oligonucleotides encoding scrambled siRNA were 5′-GAGCGATCAGATGATCCACdTdT-3′ (SEQ. ID. NO:8) and 5′-GTGGATCATCTGATCGCTCdTdT-3′ (SEQ. ID. NO:9). All siRNA were synthesized by Norris Cancer Center MicroCore Facility. At various timepoints following transfection, whole cell lysates were prepared and subjected to Western analyses using anti-phospho-H2AX or polyclonal mono-specific chicken anti-HMGA2 antibody. Control Western analyses were performed using monoclonal anti-actin antibodies.

Treatment of HS578T cells with HMGA2 siRNA virtually blocked basal H2AX phosphorylation (FIG. 6, panel B, compare lanes 14 versus lanes 5-6). A time-dependent correlation was observed between the level of endogenous HMGA2 and the extent of H2AX phosphorylation.

### Example 11

### Silencing of HMGA2 Expression Decreases the Effect of Dox on Cell Viability

HeLa/HMGA2 and HS578T cells were transfected with 100 nM HMGA2 siRNA or scrambled siRNA for 48 hours, allowed to recover for 24 hours, and then re-seeded on a 24-well plate. Cells were then treated with Dox at concentrations ranging from 04 μM, and cell growth was measured using an MTT assay as described above.

Treatment with HMGA2 siRNA enhanced cell survival by greater than two-fold in HeLa/HMGA2 cells treated with 2-4 μM Dox (FIG. 6, panel C, left side). Treatment with HMGA2 siRNA also enhanced survival in HS578T cells treated with Dox, although this effect was slightly less dramatic than that seen in HeLa/HMGA2 cells (FIG. 6, panel C, right side).

### Example 12

### HMGA2 Expression Increases Cisplatin-Induced Growth Inhibition

HeLa, HeLa/HMGA2, HS578T, and HCC1419 cells were treated with various concentrations of cisplatin ranging from 0-14 μM, and cell survival was assessed by MTT assay as described above.

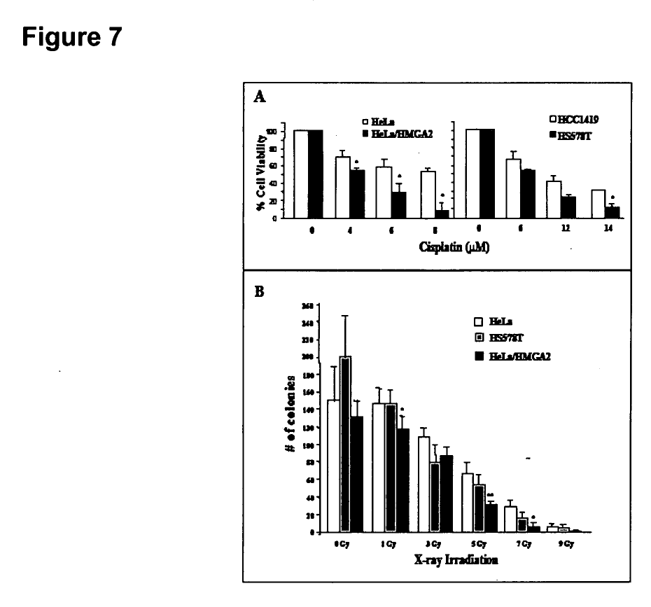

HMGA2-expressing HeLa/HMGA2 and HS578T cells were both more sensitive to cisplatin-elicited growth inhibition than HCC1419 and parental HeLa cells (FIG. 7A).

### Example 13

### HMGA2 Expression Increases Cell Radiosensitivity

HeLa, HeLa/HMGA2, and HS578T cells were exposed to ¹³⁷CS γ-irradiation at 1.55 Gy/min and irradiated with 1 to 9 Gy, returned to a tissue culture incubator for 24 hours, and then harvested for clonogenic assays. Irradiated cells were resuspended in Clonogenic medium consisting of alpha-MEM supplemented with 0.9% methylcellulose, 30% fetal bovine serum, and 50 μM β-mercaptoethanol. Cells were plated in triplicate Petri dishes at 10⁵ cells/ml/dish, and cultured in a humidified 5% CO₂ incubator for 7 days. Cancer cell colonies were enumerated on a grid using an inverted phase microscope on high optical resolution. Cells expressing HMGA2 exhibited increased radiosensitivity relative to cells that did not express HMGA2 (FIG. 7B).

### Example 14

### HMGA2 Expression Inhibits NHEJ

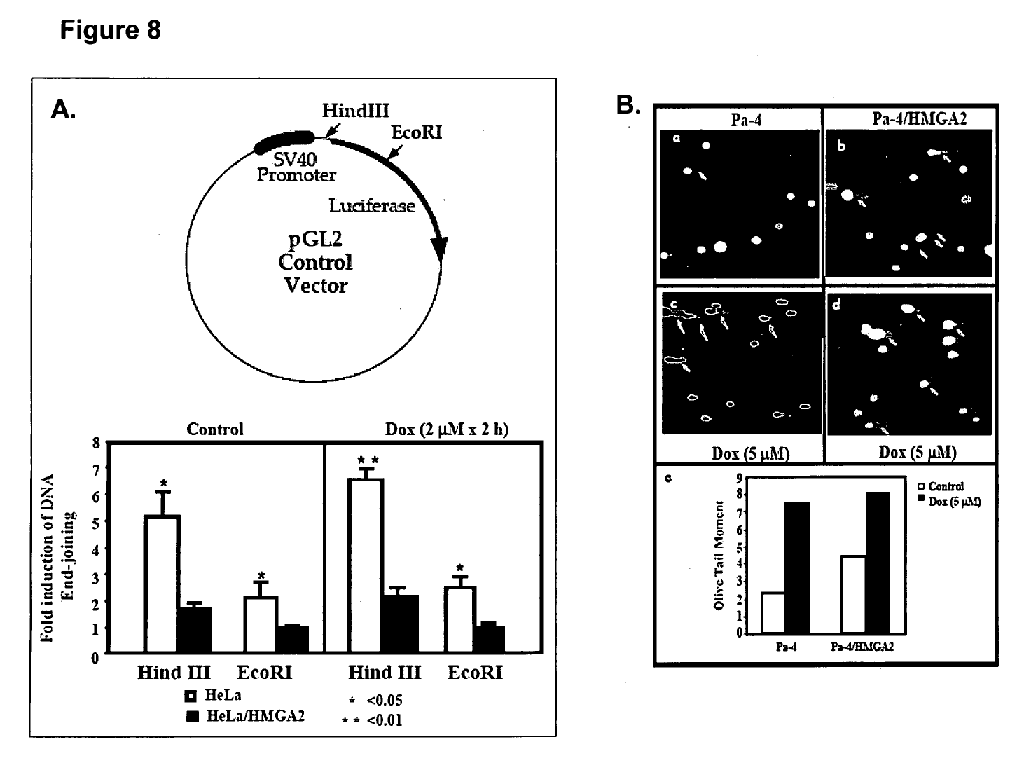

A previously reported plasmid end-joining assay (Zhong 2002) was conducted on a transiently transfected, linearized pGL2 reporter construct to determine the potential role of HMGA2 in NHEJ. The pGL2 plasmid (FIG. 8A, upper panel) was digested with either EcoRI or HindIII, verified by agarose gel electrophoresis, and purified using a Qiagen Gel Extraction kit (Qiagen, Calif.). Equal amounts of the linearized plasmid or the undigested pGL2 plasmid were transfected into Hela and Hela/HMGA2 cells together with an internal control pRL-TK luciferase plasmid using Lipofectamine 2000 (Invitrogen, CA). Transfected cells were treated with Dox or diluent for various time periods. Luciferase activity was measuring using Promega Dual-Glo Luciferase Assay (Promega, Wis.) and normalized with internal control (Renilla luciferase activity). The percentage of DNA end-joining was calculated by dividing the relative luciferase units of digested pGL2 by that of undigested pGL2.

Stable HMGA2 expression renders a decrease in overall as well as error-free end-joining activity of up to 5- and 2-fold, respectively (FIG. 8A, lower panel). The distinct NHEJ activity was also extended to Dox(2 μM)-treated HeLa/HMGA2 cells (FIG. 8A, lower panel).

### Example 15

### HMGA2 Expression is Associated with an Increase in Endogenous DNA Damage

A Comet assay (Collins 2004) was conducted to determine if HMGA2-expressing cells accumulate more DNA lesions than non-HMGA2-expressing cells prior to Dox treatment. The assay was carried out according to manufacturer protocol (Trevigen, Md.). Briefly, adherent PA-4 and PA-4/HMGA2 cells were washed and resuspended in ice-cold PBS at 1×10⁵ cells/ml before being diluted in low melting point agarose and spread onto CometSlide (Trevigen, Md.). Cells were lysed in pre-chilled Lysis Solution at 4° C. for 60 minutes and denatured with alkaline solution (12 mg/ml NaOH, 1 mM EDTA, pH>13), followed by electrophoresis at 25 V for 40 minutes. DNA was stained with SYBR green (Molecular Probes, Oreg.) and visualized using a Zeiss LSM 510 fluorescent microscope. DNA damage was quantified and scored by Olive Trail Moment using CometScore analyses software (TriTek Corp., VI).

HMGA2-expressing Pa-4 cells exhibited an approximately two-fold increase in endogenous DNA damage versus their HMGA2-underexpressing counterparts (FIG. 8B, panels b and d). This confirmed the earlier findings that HMGA2 inhibits NHEJ generally and induces accumulation of phosphorylated H2AX.

### Example 16

### Hep3B and HepG2 Cells Express Higher Levels of Endogenous HMGA2 than CL48 Cells

Immortalized liver CL48 and hepatoma Hep3B and HepG2 cells were maintained in DMEM supplemented with 10% FBS and 1% streptomycin/penicillin. HMGA2 expression levels were measured in each cell type by quantitative real-time RT-PCR. Total RNA was extracted from a 60 mm² dish using a Qiagen RNeasy Kit (Qiagen, Calif.) according to manufacturer instructions. Reverse transcription was performed with ThermoScript RT-PCR System (Invitrogen, CA). PCR reactions were set up with iQ SYBR Green Supermix (BioRad, Calif.). The sequences of the forward and reverse HMGA2 primers are set forth in SEQ ID NOs:10 and 11, respectively. The sequences of the forward and reverse primers for 18S rRNA (control gene) are set forth in SEQ ID NOs:12 and 13, respectively. Amplification and real-time quantitation were carried out using iCycler iQ Real-Time PCR Detection System (BioRad, Calif.) with the following protocol: 95° C. denaturation for three minutes, followed by 45 cycles of 15 second denaturation at 95° C. and 45 second annealing/extension at 55° C. Melt-curve analysis was performed immediately after amplification to ensure specificity of the primers and absence of primer dimers. Data were collected during each PCR cycle, and threshold cycle (CT) was calculated at the end of each run. Relative expression level was calculated with delta-delta CT method (Livak 2001; Pfaffl 2001) or comparative CT method, where the CT of the reference gene, 18S rRNA, was subtracted from that of the target gene.

Consistent with previous reports (Patel 1994), HepG2 and Hep3B cells expressed higher levels of endogenous HMGA2 than CL48 cells.

### Example 17

### HMGA2 Expression Increases Sensitivity of HepG2 and Hep3B to Dox and Cisplatin

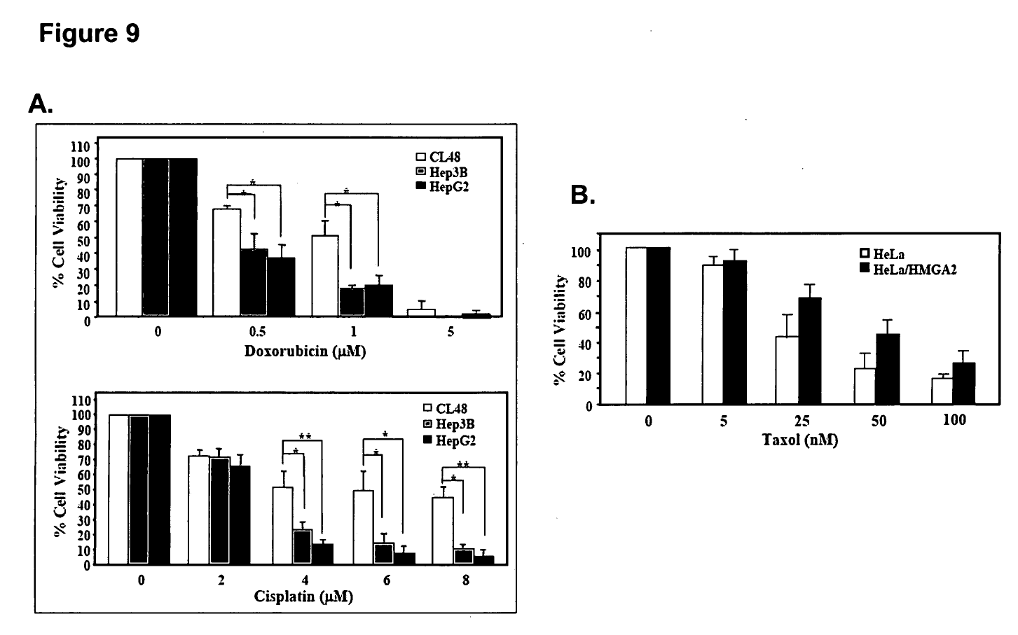

MTT assays were conducted as described above to assess the effect of HGMA2 expression on the sensitivity of HepG2, Hep3B, and CL48 cells to Dox or cisplatin. As expected, HepG2 and Hep3B cells with their higher endogenous HMGA2 levels displayed greater sensitivity to both Dox and cisplatin than HMGA2-underexpressing CL48 cells (FIG. 9A). To exclude the possibility that HMGA2 increases chemosensitization in general, Taxol-induced cell growth inhibition was measured in HeLa and HeLa/HMGA2 cells. Taxol is a microtubule stabilizing agent (Slichenmyer 1991) commonly used as a chemotherapeutic agent for combating breast and ovarian cancers. There was no substantial difference in the response to Taxol between HeLa and HeLa/HMGA2 cells (FIG. 9B), indicating that the observed HMGA2-mediated chemosensitization is restricted to agents that cause DSBs.

### Example 18

### HMGA2 Expression is Associated with DNA-PKcs Phosphorylation Following Dox Exposure

Dox treatment has been reported to lead to rapid and transient phosphorylation of DNA-PKcs at Thr-2609 (pT2609) and Ser-2056 (pS2056), respectively (Chen 2005). Equal amounts of protein lysates from HeLa and HeLa/HMGA2 cells were treated with 2 μM or 5 μM Dox, then subjected to Western analyses using polyclonal anti-phospho-DNA-PKcs-T2609/S2056 (Dr. David Chen, Univ. of Texas Southwestern Medical Center) and monoclonal anti-DNA-PKcs antibodies (Santa Cruz, Calif.). Control lysates were treated with vehicle only. Untreated and Dox-treated MO59K (DNA-PKcs proficient) and MO59J (DNA-PKcs deficient) cells were used as an additional control to assure antibody specificity.

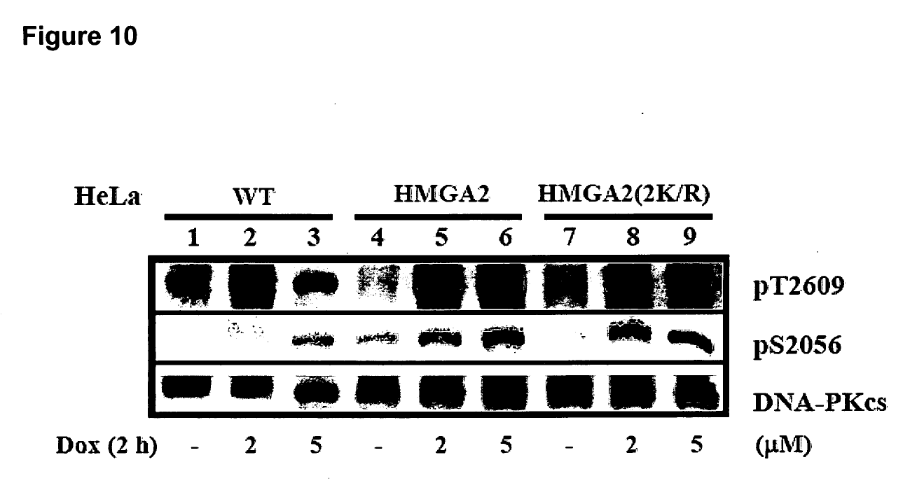

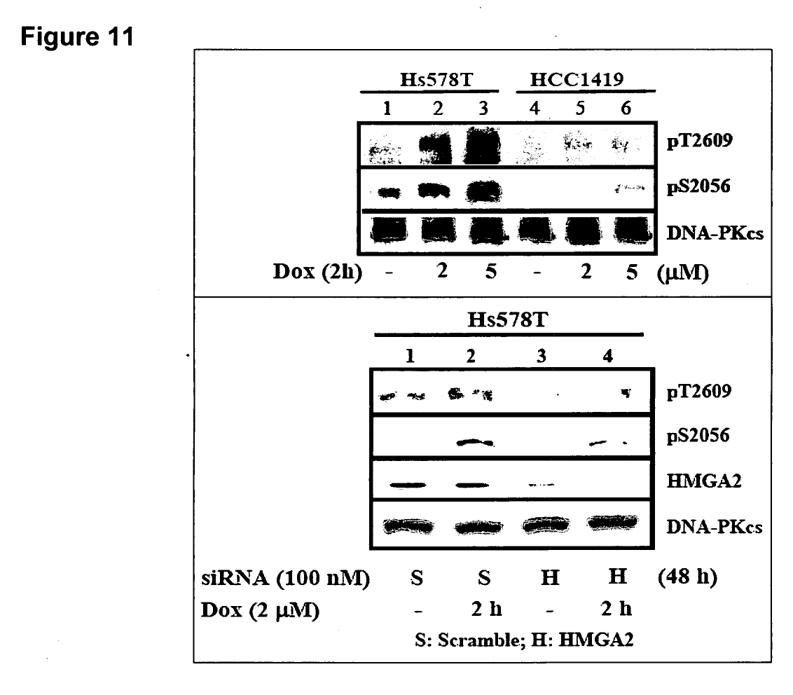

Accumulation of DNA-PKcs phosphorylation signals with no effect on DNA-PKcs steady-state levels was observed at two hours in HeLa/HMGA2 cells (FIG. 10). This accumulation was recapitulated in HMGA2-expressing Hs578T breast cancer cells, but not in HMGA2-underexpressing HCC1419 breast cancer cells (FIG. 11, top panel).

To confirm that HMGA2 expression is at least in part responsible for sustained DNA-PKcs pT2609 and pS2056 signals following Dox exposure, siRNA was utilized to downregulate endogenous HMGA2 expression in Hs578T cells. siRNA against HMGA2 (si-HMGA2) was described previously (Boo 2005). The sense and antisense were annealed to a working concentration of 20 μM by incubation with annealing buffer (5×: 30 mM HEPES pH 7.4, 100 mM potassium acetate, 2 mM magnesium acetate) at 90° C. for one minute followed by incubation at 37° C. for one hour. The annealed siRNA was stored at −20° C. Transfection of siRNA was performed with GeneEraser (Stratagene, Calif.) according to manufacturer's instruction. Briefly, cells were seeded to a confluency of 50% on the day of transfection. GeneEraser was pre-incubated at room temperature in serum-free medium ten minutes before the addition of siRNA. The GeneEraser/si-HMGA2 solution was then incubated at room temperature for an additional 15 minutes before addition to cells in a serum-containing medium. The cells were allowed to incubate for various time periods without any medium change post-transfection. Control cells were transfected with a scrambled siRNA. Cells were treated with Dox 48 hours after transfection.

si-HMGA2 conferred a decrease of pT2609- and pS2056-DNA-PKcs signals as compared to that in Hs578T cells transfected with control siRNA following Dox exposure (FIG. 11, lower panel, lanes 3 and 4 versus lanes 1 and 2).

Quantitative fluorescent immunostaining was performed to determine whether HMGA2 expression confers accumulation of pT2609-DNA-PKcs nuclear foci following Dox treatment. After Dox treatment, HeLa and HeLa/HMGA2 cells were washed with PBS and fixed in 4% paraformaldehyde at 37° C. for 15 minutes, washed three times with PBS, and permeabilized with 10% Triton-X 100 for ten minutes. The fixed cells were blocked with 2% BSA for 30 minutes and stained with primary antibodies for one hour at room temperature. After washing with PBS four times, the cells were counter-stained with secondary FITC-conjugated antibodies. Fluorescent signals were visualized using a Zeiss LSM 510 fluorescent microscope. The average volume of each DNA-PKcs-T2609 foci in approximately 75-100 cells was quantified after reconstruction in each cell line and expressed as mean foci volume. The average number of foci per cell was calculated in 75-100 cells and expressed as mean foci number per cell. Cells stained with secondary conjugated antibodies only were used as controls for background fluorescence.

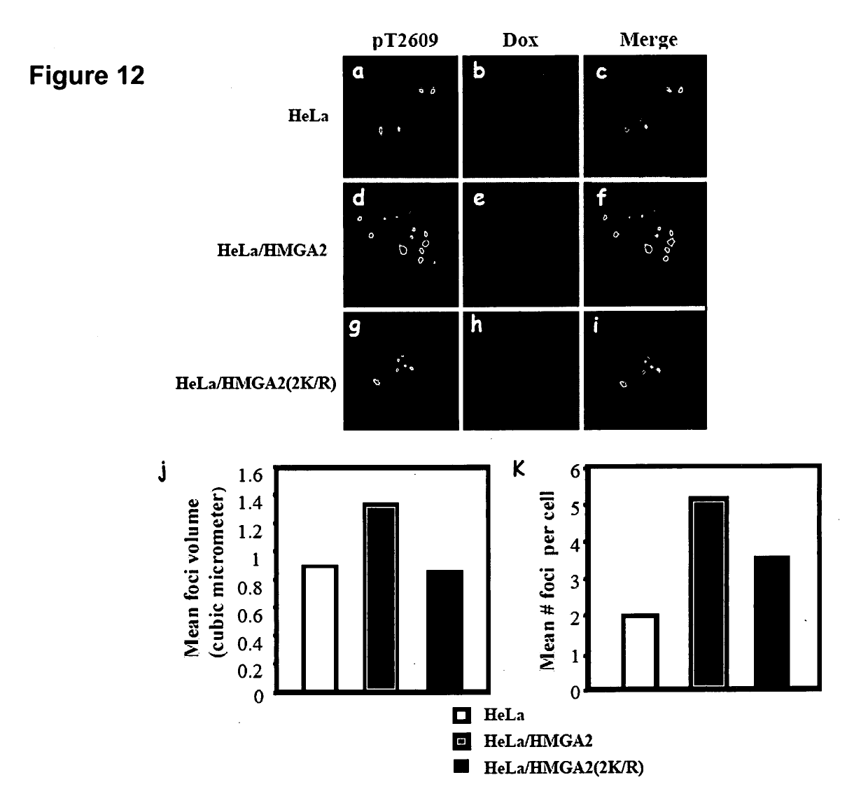

Expression of HMGA2 was accompanied by an increase in the average number and average volume of discrete nuclear pT2609-DNA-PKcs foci at 30 minutes after Dox treatment (FIG. 12, panels j and k).

### Example 19

### HMGA2-Dependent Chemosensitization is Associated with HMGA2 SUMOylation

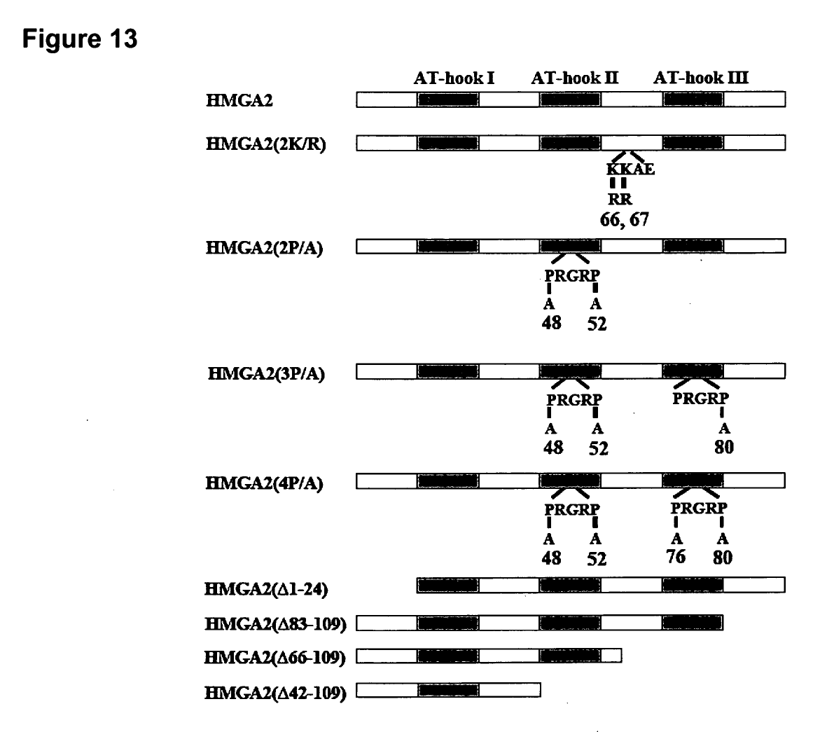

HMGA2 is a SUMOylation target (Cao, X. and Ann, D. K., unpublished observation). To investigate whether SUMOylation is involved in HMGA2-mediated chemosensitization, an HMGA2 mutant was constructed that carried Lysine to Arginine (Lys→Arg) point mutations at residues 66 and 67. These residues correspond to the SUMOyalation site (FIG. 13). The construct (HMGA2(2K/R)) was generated using previously described HA-HMGA2/pcDNA3.1 (Zentner 2001) as a template in conjunction with GeneEditor™ in vitro Site-Directed Mutagenesis System (Promega) and the mutagenesis primer set forth in SEQ ID NO:14. Desired mutations were confirmed by DNA sequencing analyses.

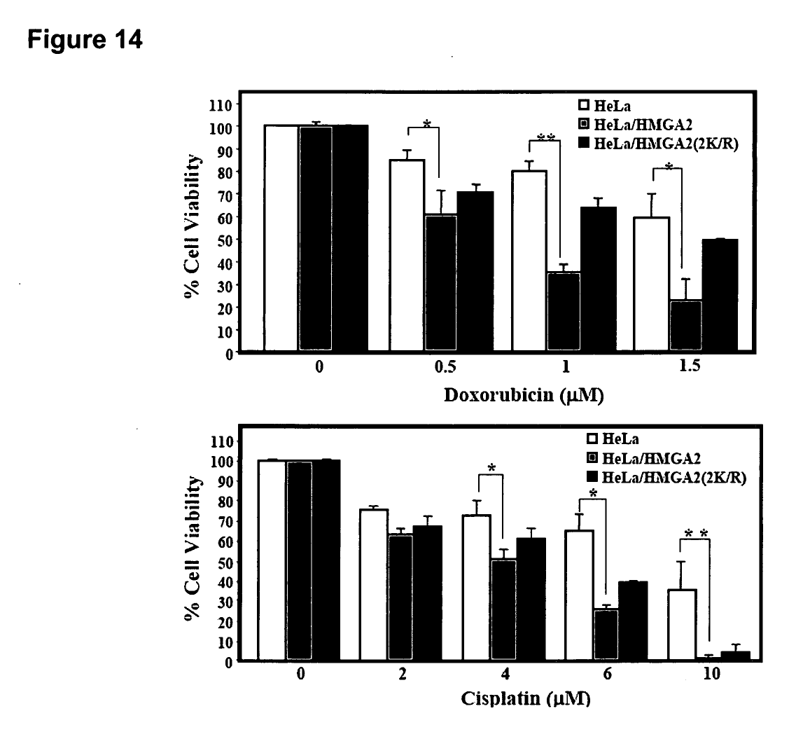

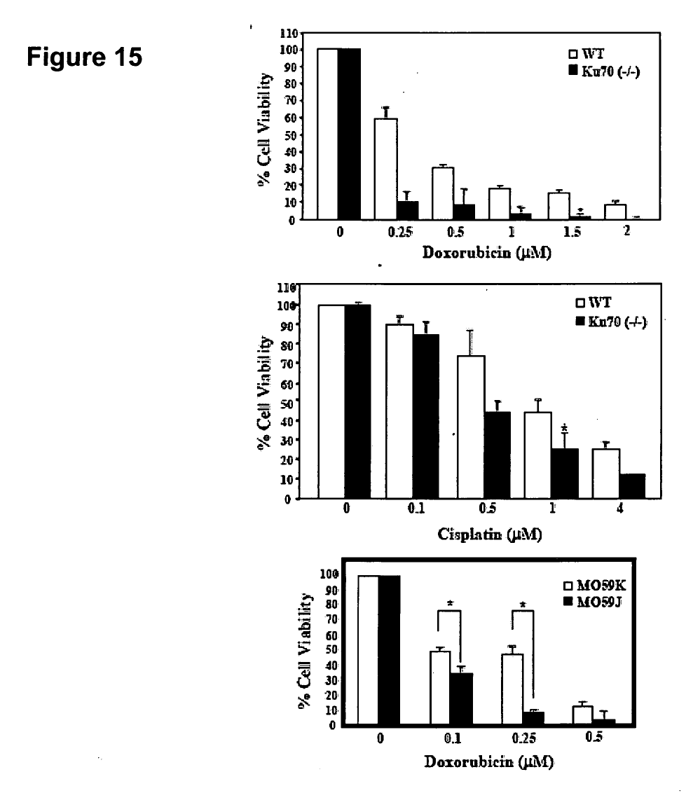

Stable expression of SUMOylation-defective HMGA2(2K/R) partially reversed the cytotoxicity elicited by Dox (FIG. 14, upper panel) and cisplatin (FIG. 15, lower panel), indicating that the SUMOylation process may play a role in mediating chemosensitization towards DSBs elicited by HMGA2.

Western analyses and quantitative fluorescent immunostaining were carried out using the same protocol described in the Example 18. Consistent with the results set forth in FIG. 11, there were fewer pT2609-DNA-PKcs signals observed in Dox-treated HeLa/HMGA2(2K/R) cells (FIGS. 10 and 12). In addition, based on the comparable accumulation of Dox-associated red fluorescence observed in the nuclei of wild-type engineered HeLa cells (FIG. 12, panels b, e, and h), it is clear that HMGA2-mediated chemosensitization towards Dox is not due to increased accumulation of imported Dox in the nucleus.

### Example 20

### HMGA2-Dependent Chemosensitization Involves DNA-PK

To confirm the involvement of DNA-PK in mediating HMGA2 chemosensitization to Dox-induced DNA damage, MTT assays were conducted as described above on Ku70-deficient (Ku70⁻/⁻) MEF cells and their wild-type counterparts and on DNA-PKcs-deficient MO59J cells and DNA-PKcs-proficient MO59K cells.

Ku70⁻/⁻ cells exhibited a marked decrease in viability following Dox (FIG. 15, upper panel) or cisplatin (FIG. 15, middle panel) treatment compared to their wild type counterparts. A similar increase in cytotoxicity was observed in DNA-PKcs-deficient MO59J cells following Dox treatment compared to DNA-PKcs-proficient MO59K cells (FIG. 15, lower panel).

### Example 21

### HMGA2 Interacts with Ku80 Via a Direct Protein-Protein Interaction

To determine whether HMGA2 directly interacts with either subunit of the DNA-PK complex to dysregulate DNA-PK activation, co-immunoprecipitation assays were conducted using an anti-Ku70 antibody in HeLa and HeLa/HMGA2 cells. Cells were lysed for one hour in RIPA buffer (PBS, 1% NP40, 0.5% sodium deoxycholate, 0.1% SDS) supplemented with PMSF, aprotinin, and sodium orthovanadate immediately before use. The cell lysates were centrifuged at 10,000 rpm for 10 minutes and supernatants were collected. Protein concentrations were determined using Bradford protein assay. Equal amounts of protein lysates were subjected to immunoprecipitation using anti-Ku70 antibodies for 2 hours at 4° C., and 20 μl of protein A/G plus (Santa Cruz, Calif.) were added and allowed to incubate overnight at 4° C. on a rotator. Samples were then analyzed by Western analyses.

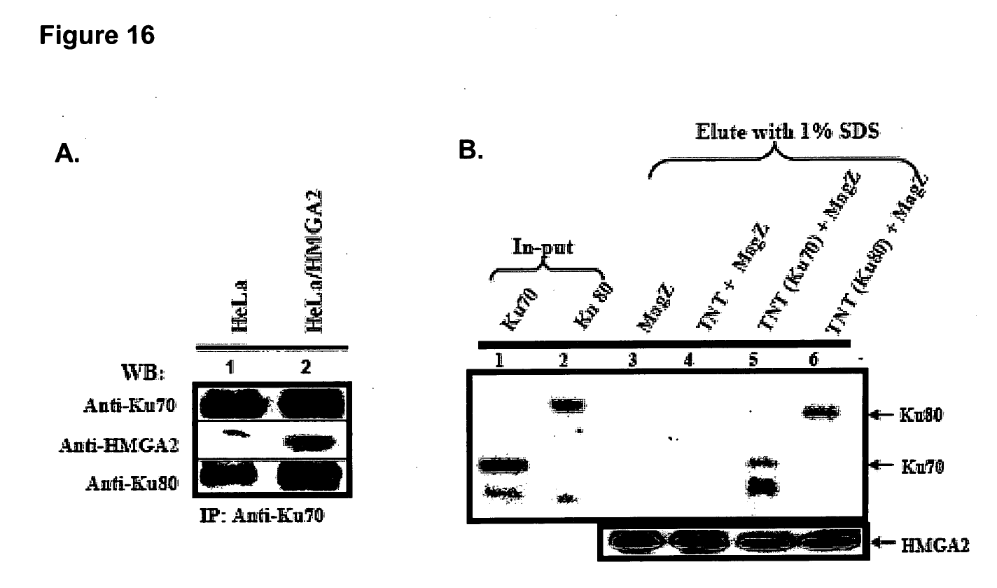

As expected, Ku80 was co-immunoprecipitated with Ku70 (FIG. 16A, lane 1). HMGA2 was also detected in the immunocomplexes of Ku80/Ku70 (FIG. 16A, lane 2).

In vitro pull-down experiments were conducted to confirm these results. Ku70/Ku80 proteins were individually translated in vitro using rabbit reticulocyte lysates of the TNT Quick Coupled Transcription/Translation Systems with Transcend Chemiluminescent detection system (Promega, Wis.). 500 μl of the overnight culture of pET21 b-His-HMGA2 in LB media supplemented with 0.1 mg/ml ampicillin was repopulated in 10 ml LB media supplemented with ampicillin. The bacterial culture was grown at 37° C. with shaking (225 rpm) until OD₆₀₀ reached 0.5-0.7, at which point 1 mM IPTG was added to the bacterial culture to induce protein expression. After 3 hours, proteins were harvested by lysing the bacteria with 1 ml of Fast-Break Lysis Buffer (Promega, Wis.). 100 μl of the lysed bacteria was added to 30 μl of MagZ particles (Promega, Wis.) and incubated for 15 minutes at room temperature. A magnetic stand was used to capture the MagZ particles while the supernatant was carefully removed. MagZ particles were washed three times with MagZ Bind/Wash buffer (Promega, Wis.). After the final wash, the particles were resuspended in 175 μl of MagZ Bind/Wash buffer. 20 μl of TNT translated Ku70/Ku80 reactions were added to the mixture, respectively, and incubated for 1 hour at room temperature on a shaker. The mixture was washed three times with MagZ Bind/Wash buffer and the bound proteins were eluted with 1% SDS. The proteins were separated on SDS-PAGE, transferred onto a PVDF membrane, and visualized with streptavidine-HRP.

The results showed that in vitro individually translated Ku80 was able to be pulled down by HMGA2, while Ku70 alone was bound to a lesser extent (FIG. 16B). This verified that the interaction between Ku80 and HMGA2 occurs at least in part via a direct protein-protein interaction in vitro.

### Example 22

### HMGA2 Dissociates from the Ku70/Ku80 Complex Following Dox Treatment

To determine whether Dox treatment affects the protein-protein interaction between HMGA2 and Ku80/Ku70, HeLa and HeLa/HMGA2 cells were exposed to a single dose of 10 μM Dox for 30 minutes. Cell lysates were then subjected to immunoprecipitation assays with an anti-HA tag (for HMGA2) or an anti-Ku80 antibody using the protocol described in Example 21.

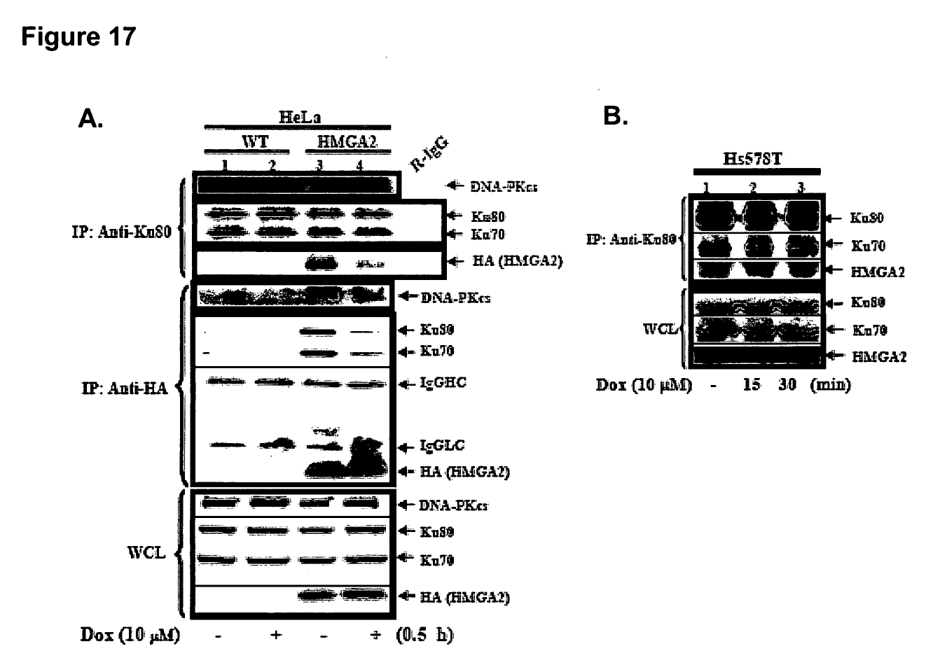

Dox treatment enriched the amount of DNA-PKcs detected in the immunocomplexes of Ku80 prepared from Dox-treated HeLa/HMGA2 cells (FIG. 17A, upper panel), supporting the idea that HMGA2 potentiates Dox-induced DSBs as more DNA-PKcs being recruited to the sites of Ku80/Ku70-bound DSBs. HA-HMGA2 was also detected in the immunocomplexes of Ku80. However, Dox-treatment rendered a decrease in anti-Ku80-immunoprecipitated HMGA2 without affecting the steady-state level of Ku70 detected in the Ku80-immunocomplexes in HeLa/HMGA2 cells (FIG. 17A, upper panel).

The effect of Dox treatment on the interaction between HMGA2 and the Ku70/Ku80 heterodimer was further confirmed by a reciprocal co-immunoprecipitation assay using an anti-HA antibody (HMGA2) (FIG. 17A, middle panel). Interestingly, DNA-PKcs were detected in the immunocomplexes of HMGA2 (FIG. 17A, middle panel) prior to Dox treatment in HeLa/HMGA2 cells, supporting the notion that HMGA2 promotes an accumulation of endogenous DNA DSBs (FIGS. 8A, 8B, 10, and 12). To exclude the possibility that the observed HMGA2/Ku80/Ku70 interaction was an artifact caused by engineered overexpression of HMGA2 in HeLa/HMGA2 cells, the interaction between endogenous HMGA2 and Ku80/Ku70 in Hs578T breast cancer cells was examined. As shown in FIG. 17B, endogenous HMGA2 interacted with Ku80/Ku70, and this interaction was disrupted upon Dox-treatment. Together, these results suggest that HMGA2 dysregulates DNA damage response by forming complexes with Ku80/Ku70 and/or DNA-PKcs prior to Dox treatment, and dissociates from such complexes following Dox exposure.

### Example 23

### HMGA2 Interacts with Ku80 Via Protein-DNA Interactions

Three different truncated forms of HMGA2 were constructed carrying either deletions of the N-terminus (Δ1-24), C-terminus (Δ83-109), or C-terminus with the AT-hook III (Δ66-109) (FIG. 13). To generate these constructs, three primer pairs were designed and synthesized according to HMGA2 sequences and appropriate restriction enzyme sites foe subsequent cloning purpose. These primer pairs are set forth SEQ ID NOs:15-16 (HA-HMGA2(Δ1-24), 17-18 (HA-HMGA2(Δ83-109), and 19-20 (HA-HMGA2(Δ66-109), respectively. These primer pairs were used to perform individual PCR with pcDNA3-HA-HMGA2. The PCR product was digested with corresponding enzymes and swapping with HA-HMGA2 in pcDNA3-HA-HMGA2 to create the desired variants. Co-immunoprecipitation assays were performed following transient transfection of HeLa cells with these constructs.

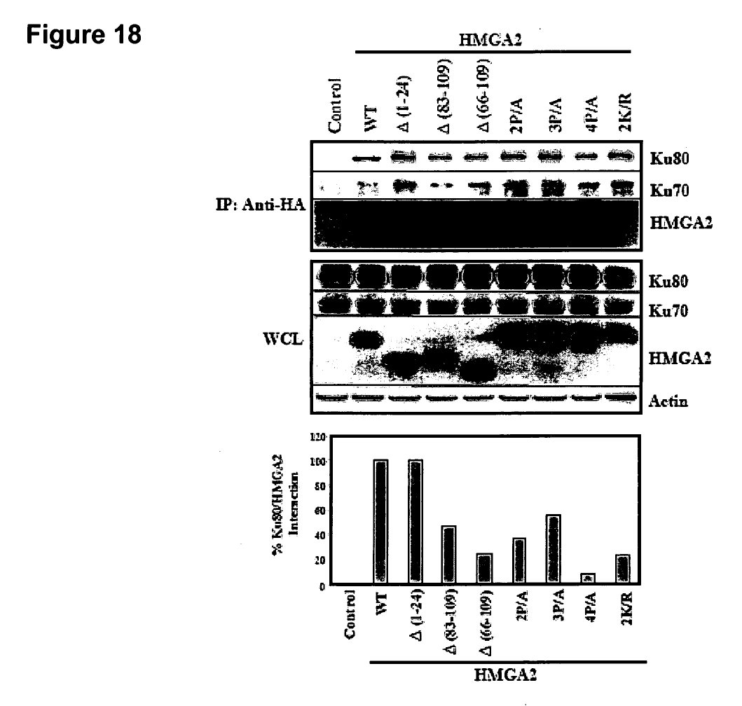

The only mutant HMGA2 that retained the full ability to interact with Ku80 was HMGA2(Δ1-24) (FIG. 18). The interaction decreased by more than 70% in HMGA2(Δ66-109) and by more than 50% in HMGA2(Δ83-109).

A deletion mutant of the C-terminus with the AT-hook II and III was created (FIG. 13). The primer pair used to generate this construct (HMGA2(Δ42-109)) is set forth in SEQ ID NOs:21-22. This mutant failed to express after transient transfection.

To further investigate whether the DNA binding ability of the AT-hooks in HMGA2 is necessary for its interaction with Ku80, an HMGA2 mutant (HMGA2(4P/A)) was generated harboring four Pro→Ala mutations at amino acid positions 48, 52, 76, and 80 (FIG. 13). These residues, located at the second and third DNA-binding AT-hooks of HMGA2, are reported to abolish the DNA binding ability of HMGA (Himes 2000). The two mutagenesis primers used to generate the HMGA2(4P/A) construct are set forth in SEQ ID NOs:23-24. SEQ ID NO:23 rendered the P48A and P53A mutations, while SEQ ID NO:24 rendered the P76A and P80A mutations. The four mutations almost completely abrogated the binding of HMGA2 to Ku80 (FIG. 18, upper panel).

To map more precisely the DNA binding region within HMGA2 involved in complex formation, HMGA2(2P/A) and HMGA2(3P/A) were constructed, harboring Pro→Ala mutations at amino acid positions 48/52 (AT-hook II) and 48/52/80 (AT-hook II and III), respectively. Results show that the Pro residues in the second AT-hook of HMGA2 appear to be essential to the HMGA2 interaction with Ku80 (FIG. 18, upper panel). The relative ability of each HMGA2 mutant to interact with Ku80 after normalizing for transfection efficiency and protein loading is set forth in the lower panel of FIG. 18. These results, taken together with those in FIG. 16B, show that HMGA2 is likely to complex with Ku80 via both protein-protein and protein-DNA interactions.

### Example 24

### Complex Formation between HMGA2 and Ku80 Contributes to HMGA2-Mediated Dox Chemosensitization

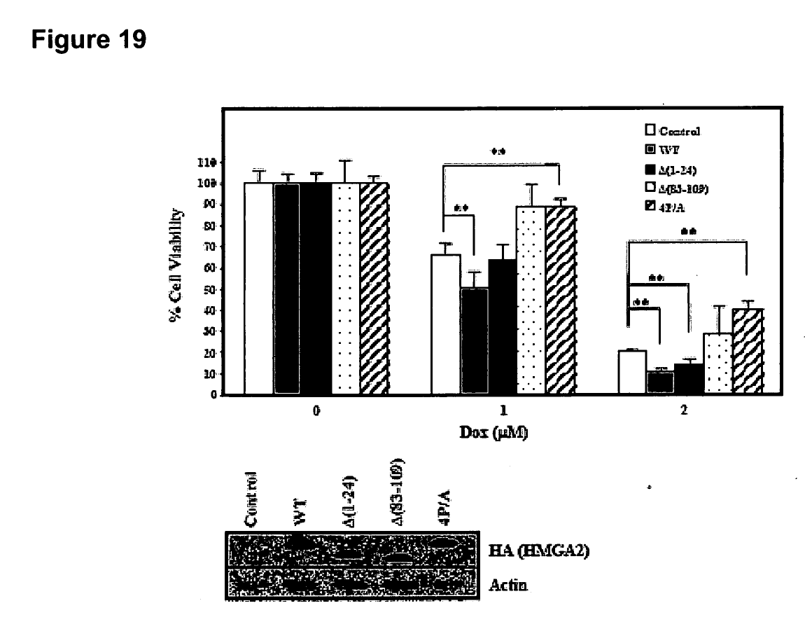

To elucidate the relevance of HMGA2 complex formation with Ku80/Ku70, the viability of Dox-treated HeLa cells transfected with HMGA2 or its engineered mutants was examined. Transient transfection of HMGA2 in HeLa cells increased their sensitivity towards Dox, consistent with previous results using cells with stable HMGA2 expression of HMGA2 (FIG. 19, upper panel). Moreover, transfection with HMGA2(Δ1-24), which interacts with Ku80/Ku70 in a manner comparable to that of wild-type HMGA2 (FIG. 18), enhanced the sensitivity of HeLa cells to Dox. By contrast, HeLa cells expressing HMGA2(Δ83-109) or HMGA2(4P/A) were exhibited relatively greater resistance to Dox-treatment. The change in their individual sensitivity to Dox was not a result of different expression level from each transfected HMGA2, since all forms showed comparable expression (FIG. 19, lower panel). The augmented resistance to Dox, relative to that of vector-transfected HeLa cells, by HMGA2(Δ83-109) and HMGA2(4P/A) mutants may be due to protein-protein interactions arising from the remaining portion of HMGA2. Taken together, these results suggest that the ability of HMGA2 to complex with Ku80 contributes, at least in part, to the HMGA2-mediated Dox chemosensitization.

### Example 25

### DNA-PKcs Function is Required for Dox-Induced pSer1981-ATM and pThr68-Chk2 Signal

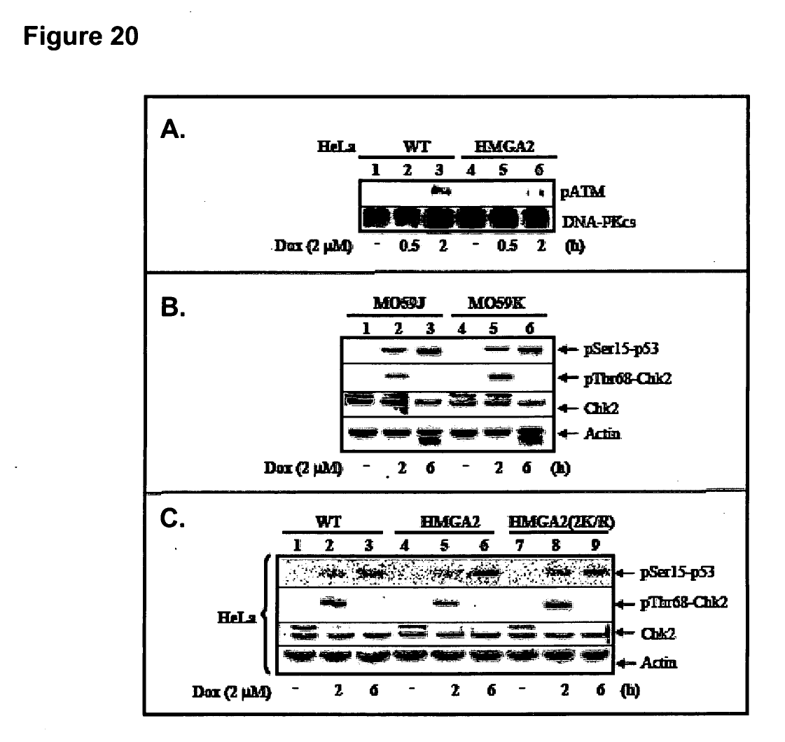

Based on the reciprocal dependency between DNA-PK and ATM (Chan 2002; Peng 2005), experiments were performed to determine whether the sustained DNA-PKcs phosphorylation profile is accompanied with the dysregulated DNA damage-triggered signaling of other PIKK members. The ATM activation profile of HeLa cells and HeLa/HMGA2 cells in response to Dox treatment was probed. The results indicated that HMGA2 delayed ATM Ser-1981 activation upon exposure to 2 μM Dox (FIG. 20A).

Both DNA-PK and ATM have been shown to initiate DNA damage signaling by enhancing phosphorylation of p53 at Ser-15 and Chk2 at Thr-68 (Wang 2000; Lukas 2003). To establish the role of DNA-PK in Dox-induced p53 and Chk2 phosphorylation, MO59J (DNA-PKcs-deficient) and MO59K (DNA-PKcs-proficient) cells were treated with Dox, and their p53 and Chk2 phosphorylation profiles were probed. Dox-treatment rendered a robust p53 phosphorylation at Ser-15 (pSer15-p53) in both MO59J and MO59K cells (FIG. 20B). By contrast, Chk2 phosphorylation at Thr-68 (pThr68-Chk2) was barely detectable in MO59J cells at 2 hours post-Dox treatment (FIG. 20B, lane 2 versus lane 5). Together, these results suggest that proper DNA-PKcs function is required for Dox-induced pSer1981-ATM (FIG. 20A) and pThr68-Chk2 (FIG. 20C) signal, but not pSer15-p53 signal (FIG. 20B).

### Example 26

### HMGA2 Disrupts the DNA Damage Response by Interfering with the Activation of DNA-PKcs, ATM, and Chk2

pSer15-p53 and pThr68-Chk2 profiles following exposure to Dox in parental and engineered HeLa cells were assessed. Exposure of all cells to Dox caused induction of pSer15-p53 and pThr68-Chk2 signals (FIG. 20C). Intriguingly, there was a noticeable accumulation of pSer15-p53 at 6 hours post-treatment and attenuation of pThr68-Chk2 at 2 hours post-treatment in HeLa/HMGA2 cells, compared with Dox-treated HeLa and HeLa/HMGA2(2K/R) cells. Together with results set forth in Example 25, HMGA2 appears to disrupt DNA damage response by interfering with the activation of DNA-PKcs, ATM and Chk2.

### Example 27

### HMGA2 Expression Facilitates Dox-Induced Caspase-3 Activation and p53 Expression

To determine whether HMGA2 expression facilitates caspase-3 and/or p53 activation, protein lysates from Dox treated HeLa and HeLa/HMGA2 cells were subjected to Western analyses using anti-caspase-3 and anti-p53 antibodies. Anti-tubulin antibodies were used as a control.

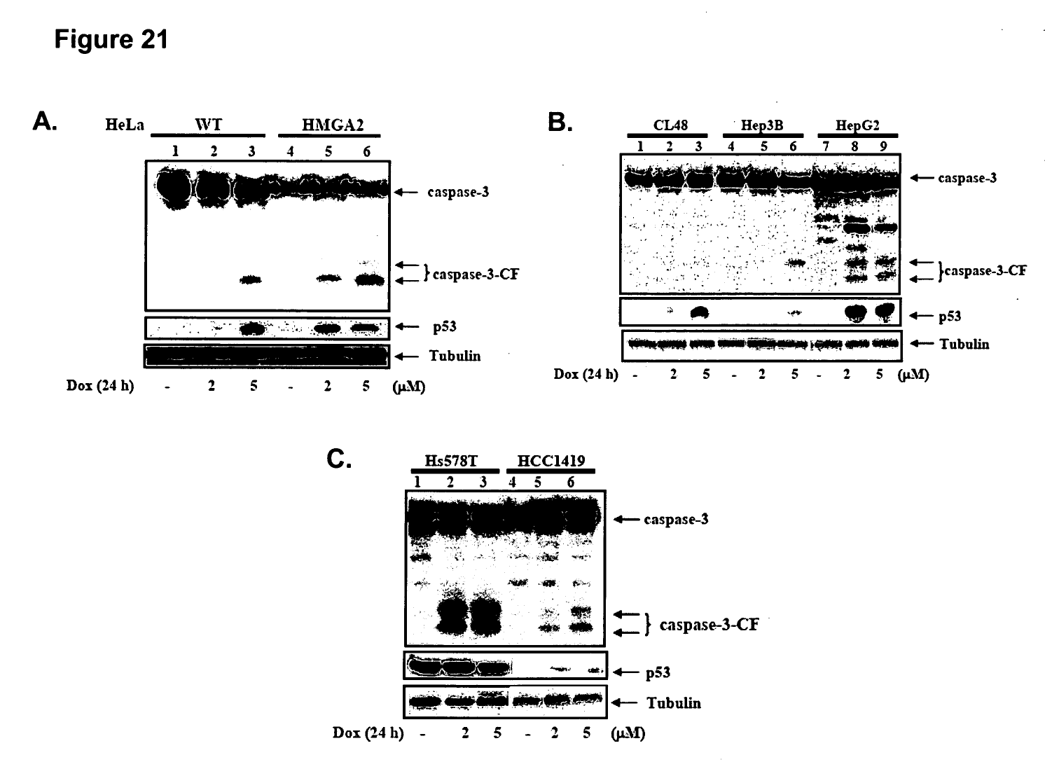

Generation of caspase-3 cleavage fragment (caspase-3-CF), a hallmark of caspase-3 activation, was increased in HeLa/HMGA2, HepG2 and Hs578T cells following treatment with Dox (FIG. 21). This increase correlated with chemosensitization of the cells. Dox treatment also increased steady-state p53 levels in HeLa, HeLa/HMGA2, CL48, and HepG2 cells, with Hep3B cells serving as a p53-null control (FIG. 21).

### Example 28

### HMGA2 Expression Does Not Enhance Dox-Induced PUMA and NOXA Transcriptional Activation

Expression of the BH3-only proteins PUMA and NOXA is reported to be transcriptionally simulated by p53 following DNA damage (Villunger 2003; Yakovlev 2004; Chipuk 2005). Quantitative real-time RT-PCR experiments were conducted to determine the effect of HMGA2 expression on PUMA and NOXA transcriptional activation following Dox treatment. The primer pairs used to analyze relative mRNA levels of PUMA and NOXA are set forth in SEQ ID NOs: 25-26 and 27-28, respectively.

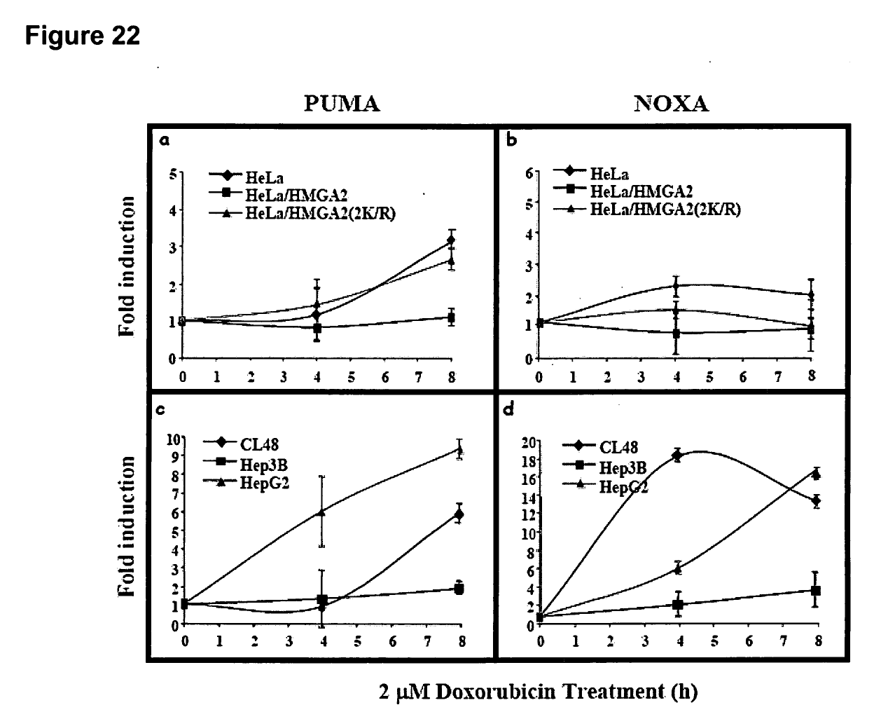

Dox induced marked transcriptional activation of PUMA and NOXA at 8 hours after treatment in CL48 and HepG2 cells (FIG. 22, panels c and d), but there was a modest induction if any in Hep3B cells and all HeLa cells tested (FIG. 22). Thus, Dox treatment induces PUMA and NOXA expression in PUMA and NOXA cells, but PUMA and NOXA expression does not correlate with the decreased viability and enhanced activation of caspase-3 observed in HMGA2-expressing cells following Dox treatment (FIGS. 9A, 14, and 21).

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Abdulkadir, S. A., et al. 1995. J Exp Med 182:487-500.
2. Abe, N., et al. 2003. Br J Cancer 89:2104-2109.
3. Abraham, R. T. 2001. Genes Dev 15:2177-2196.
4. Abraham, R. T. 2004. DNA Repair (Amst) 3:883-887.
5. Adair, J. E., et al. 2005. J Biol Chem 280:32184-32192.
6. Akai, T., et al. 2004. Pathol Res Pract 200:619-624.
7. Al Rashid, S. T., et al. 2005. Cancer Res 65:10810-10821.
8. Alt, J. R., et al. 2005. J Biol Chem 280:18771-18781.
9. Anand, A., Chada, K. 2000. Nat Genet 24:377-380.
10. Ashar, H. R., et al. 1995. Cell 82:57-65.
11. Baldassarre, G., et al. 2005. Oncogene 24:6809-6819.
12. Bartkova, J., et al. 2005. Nature. 434:864-870.
13. Berlingieri, M. T., et al. 1995. Mol Cell Biol 15:1545-1553.
14. Block, W. D., et al. 2004. Nucleic Acids Res 32:4351-4357.
15. Boo, L. M., et al. 2005. Cancer Res 65:6622-6630.
16. Boubnov, N. V., et al. 1995. Proc Natl Acad Sci USA 92:890-894.
17. Bryant, H. E., et al. 2005. Nature 434:913-917.
18. Burma, S., et al. 2001. J Biol Chem 276:42462-42467.
19. Burma, S., Chen, D. J. 2004. DNA Repair (Amst) 3:909-918.
20. Celeste, A., et al. 2002. Science 296:922-927.
21. Celeste, A., et al. 2003. Nat Cell Biol 5:675-679.
22. Chan, D. W., et al. 2002. Genes Dev 16:2333-2338.
23. Ch'ang, H. J., et al. 2005. Nat Med 11:484-490.
24. Chau, K. Y., et al. 2000. J Neurosci 20:7317-7324.
25. Chen, B. P., et al. 2005. J Biol Chem 280:14709-14715.
26. Chin, M. T., et al. 1998. J Biol Chem 273:9755-9760.
27. Chipuk, J. E., et al. 2005. Science 309:1732-1735.
28. Collins, A. R. 2004. Mol Biotechnol 26:249-261.
29. Cortez, D. 2003. J Biol Chem 278:37139-37145.
30. Cory, S., Huang, D. C., Adams, J. M. 2003. Oncogene 22:8590-8607.
31. Couedel, C., et al. 2004. Genes Dev 18:1293-1304.
32. De Silva, I. U., McHugh, P. J., Clingen, P. H., Hartley, J. A. 2000. Mol Cell Biol 20:7980-7990.
33. Diana, F., et al. 2005. Int J Oncol 26:515-520.
34. Ding, Q., et al. 2003. Mol Cell Biol 23:5836-5848.
35. Douglas, P., et al. 2002. Biochem J 368:243-251.
36. Durocher, D., Jackson, S. P. 2001. Curr Opin Cell Biol 13:225-231.
37. Farmer, H., et al. 2005. Nature 434:917-921.
38. Featherstone, C., Jackson, S. P. 1999. Mutat Res 434:3-15.
39. Fedele, M., et al. 2001. Carcinogenesis 22:1583-1591.
40. Fedele, M., et al. 2002. Oncogene 21:3190-3198.
41. Fedele, M. et al. 2006. Cancer Cell 9:459-471.
42. Fejzo, M. S., et al. 1995. Genomics 26:265-271.
43. Finelli, P., et al. 2002. Cancer Res 62:2398-2405.
44. Geske, F. J., et al. 2000. Cell Death Differ 7:393-401.
45. Gorgoulis, V. G., et al. 2005. Nature 434:907-913.
46. Guirouilh-Barbat, J., et al. 2004. Mol Cell 14:611-623.
47. Hall-Jackson, C. A., Cross, D. A., Morrice, N., Smythe, C. 1999. Oncogene 18:6707-6713.
48. Hannon, G. J. 2002. Nature 418:244-251.
49. Himes, S. R., et al. 2000. J Immunol 164:3157-3168.
50. Hirning-Folz, U., et al. 1998. Genes Chromosomes Cancer 23:350:357.
51. Hong, S., Pusapati, R. V., Powers, J. T., Johnson, D. G. 2006. Cell Cycle 5:801-803.
52. Hornung, V., et al. 2005. Nat Med 11:263-270.
53. Hsu, H. L., Yannone, S. M., Chen, D. J. 2002. DNA Repair (Amst) 1:225-235.
54. Jeggo, P. A. 1998. Adv Genet 38:185-218.
55. Jeggo, P. A., Lobrich, M. 2006. DNA Repair (Amst). Epub ahead of print.
56. Johnson, P. F. 2005. J Cell Sci 118:2545-2555.
57. Kim, D. H., et al. 2004. Nat Biotechnol 22:321-325.
58. Klampfer, L., et al. 2005. Oncogene 24:3932-3941.
59. Koch, C. A., et al. 2004. EMBO J. 23:3874-3885.
60. Kurz, E. U., Lees-Miller, S. P. 2004. DNA Repair (Amst) 3:889-900.
61. Langelotz, C., et al. 2003. Br J Cancer 88:1406-1410.
62. Lees-Miller, S. P., et al. 1995. Science 267:1183-1185.
63. Li, D., et al. 1997. J Biol Chem 272:25062-25070.
64. Li, S., et al. 2000. Nature 406:210-215.
65. Li, T. K., Liu, L. F. 2001. Annu Rev Pharmacol Toxicol 41:53-77.
66. Li, J., Stern, D. F. 2005. J Biol Chem 280:37948-37956.
67. Livak, K. J., Schmittgen, T. D. 2001. Methods 25:402-408.
68. Lukas, C., et al. 2003. Nat Cell Biol 5:255-260.
69. Ma, Y., Pannicke, U., Schwarz, K., Lieber, M. R. 2002. Cell 108:781-794.
70. Ma, Y., et al. 2004. Mol Cell 16:701-713.
71. Mahajan, K. N., et al. 2002. Mol Cell Biol 22:5194-5202.
72. Mantovani, F., et al. 1998. Nucleic Acids Res 26:1433-1439.
73. Masciullo, V., et al. 2003. Carcinogenesis 24:1191-1198.
74. Merkle, D., et al. 2002. Biochemistry 41:12706-12714.
75. Mikhailov, A., Shinohara, M., Rieder, C. L. 2004. J Cell Biol 166:517-526.
76. Minotti, G., et al. 2004. Pharmacol Rev 56:185-229.
77. Miyazawa, J., et al. 2004. Cancer Res 64:2024-2029.
78. Motoyama, N., Naka, K. 2004. Curr Opin Genet Dev 14:11-16.
79. Mukherjee, B., et al. 2006. DNA Repair (Amst) 5:575-590.
80. Munshi, N., et al. 2001. Science 293:1133-1136.
81. Nakano, K., Vousden, K. H. 2001. Mol Cell 7:683-694.
82. Nghiem, P, et al. 2002. J Biol Chem 277:4428-4434.
83. Nissen, M. S., Reeves, R. 1995. J Biol Chem 270:4355-4360.
84. Norbury, C. J., Zhivotovsky, B. 2004. Oncogene 23:2797-2808.
85. Noro, B., et al. 2003. Biochem 42:4569-4577.
86. Patel, U. A., et al. 1994. Biochem Biophys Res Commun 201:63-70.
87. Paull, T. T., et al. 2000. Curr Biol 10:886-895.
88. Pearce, A. K., Humphrey, T. C. 2001. Trends Cell Biol 11:426-433.
89. Peng, Y., et al. 2005. Cancer Res 65:1670-1677.
90. Pirnia, F., et al. 2002. Cell Death Differ 9:905-914.
91. Pfaffl, M. W. 2001. Nucleic Acids Res 29:e45.
92. Pickering, M. T., Kowolik, T. F. 2006. Oncogene 25:746-755.
93. Pierantoni, G. M., et al. 2005. Cell Death Differ. Epub ahead of print.
94. Pilch, D. R., et al. 2003. Biochem Cell Biol 81:123-129.
95. Reeves, R., Nissen, M. S. 1993. J Biol Chem 268:21137-21146.
96. Reeves, R. 2001a. Gene 277:63-81.
97. Reeves, R., Beckerbauer, L. 2001b. Biochim Biophys Acta 1519:13-29.
98. Riballo, E., et al. 2004. Mol Cell 16:715-724.
99. Rogakou, E. P., et al. 1998. J Biol Chem 273:5858-5868.
100. Rogakou, E. P., Boon, C., Redon, C., Bonner, W. M. 1999. J Cell Biol 146:905-916.
101. Rogalla, P., et al. 1997. Mol Carcinog 19:153-156.
102. Ross, J. S., et al. 2004. Am J Clin Pathol 122:598-609.
103. Rouse, J., Jackson, S. P. 2002. Science 297:547-551.
104. Sarhadi, V. K., et al. 2006. J Pathol 209:206-212.
105. Sarkaria, J. N., et al. 1999. Cancer Res 59:4375-4382.
106. Schoenmakers, E. F., et al. 1995. Nat Genet 10:436-444.
107. Sengupta, S., Harris, C. C. 2005. Nat Rev Mol Cell Biol 6:44-55.
108. Sgarra, R., et al. 2004. FEBS Lett 574:1-8.
109. Shiloh, Y. 2003. Nat Rev Cancer 3:155-168.
110. Shiloh, Y., et al. 2004. Semin Cancer Biol 14:295-305.
111. Sledz, C. A., et al. 2003. Nat Cell Biol 5:834-839.
112. Sledz, C. A., Williams, B. R. 2004. Biochem Soc Trans 32:952-956.
113. Slichenmeyer, W. J., Von Hoff, D. D. 1991. Anticancer Drugs 2:519-530.
114. Stewart, G. S., et al. 2003. Nature 421:961-966.
115. Tallini, G., Dal Cin, P. 1999. Adv Anat Pathol 6:237-246.
116. Tessari, M. A., et al. 2003. Mol Cell Biol 23:9104-9116.
117. Thanos, D., Maniatis, T. 1992. Cell 71:777-789.
118. Venkitaraman, A. R. 2005. Nature 434:829-830.
119. Villunger, A., et al. 2003. Science 302:1036-1038.
120. Veuger, S. J., et al. 2003. Cancer Res 63:6008-6015.
121. Wang, S., et al. 2000. Proc Natl Acad Sci USA 97:1584-1588.
122. Wang, B., Matsuoka, S., Carpenter, P. B., Elledge, S. J. 2002. Science 298:1435-1438.
123. Wang, D., Lippard, S. J. 2005. Nat Rev Drug Discov 4:307-320.
124. Wang, G. L., Timchenko, N. A. 2005. Mol Cell Biol 25:1325-1338.
125. Ward, I. M., Chen, J. 2001. J Biol Chem 276:47759-47762.
126. Watters, J. W., McLeod, H. L. 2003. Biochim Biophys Acta 1603:99-111.
127. Weizman, N., et al. 2003. J Biol Chem 278:6741-6747.
128. West, S. C. 2003. Nat Rev Mol Cell Biol 4:435-445.
129. Willmore, E., et al. 2004. Blood 103:4659-4665.
130. Yakovlev, A. G., et al. 2004. J Biol Chem 279:28367-28374.
131. Yaneva, M., Kowalewski, T., Lieber, M. R. 1997. EMBO J. 16:5098-5112.
132. Yin, H., Glass, J. 2006. J Biol Chem 281:11496-11505.
133. Zentner, M. D., et al. 2001. J Biol Chem 276:29805-29814.
134. Zhang, X. M., Verdine, G. L. 1999. J Biol Chem 274:20235-20243.
135. Zhong, Q., et al. 2002. J Biol Chem 277:28641-28647.
136. Zimmerman, E. S., et al. 2004. Mol Cell Biol 24:9286-9294.
137. Zhou, X., Benson, K. F., Ashar, H. R., Chada, K. 1995. Nature 376:771-774.
138. Zhou, X., et al. 1996. Genomic structure and expression of the murine Hmgi-c gene. Nucleic Acids Res 24:4071-4077.
139. Zhou, B. B., et al. 2000. J Biol Chem 275:10342-10348.

What is claimed is:

1. A method of predicting the likelihood that treatment with a DNA-damaging therapeutic agent will be effective in a subject with cancer comprising measuring HMGA2 expression in one or more cancer cells from said subject.

2. A method of identifying a subject with cancer who is likely to respond favorably to treatment with a DNA-damaging therapeutic agent comprising measuring HMGA2 expression levels in one or more cancer cells from said subject, wherein said subject is likely to respond favorably to said treatment if one or more of said cancer cells exhibit HMGA2 expression levels that are greater than or equal to a threshold level.

3. A method of treating cancer in a subject comprising
a) measuring HMGA2 expression levels in one or more cancer cells from said subject; and
b) if said HMGA2 expression levels are greater than or equal to a threshold level, administering to said subject a DNA-damaging therapeutic agent.

4. A kit for determining whether a subject is likely to respond favorably to treatment with a DNA-damaging therapeutic agent comprising a means for measuring HMGA2 expression levels in one or more cancer cells from said subject, wherein a subject is likely to respond favorably to said treatment if the HMGA2 expression levels in one or more of said cancer cells are greater than or equal to a threshold value.

5. A method of increasing the sensitivity of a cancer cell to treatment with a DNA-damaging therapeutic agent comprising introducing into said cell a polynucleotide encoding HMGA2 or a fragment thereof.

6. The method of claim 5, wherein said DNA-damaging therapeutic agent is selected from the group consisting of doxorubicin, cisplatin, and X-ray irradiation.

7. A method of increasing the effectiveness of treatment with a DNA-damaging therapeutic agent in a subject with cancer comprising administering to said subject a therapeutically effective amount of an HMGA2 polypeptide or a fragment thereof.

8. The method of claim 7, wherein said DNA-damaging therapeutic agent is selected from the group consisting of doxorubicin, cisplatin, and X-ray irradiation.


SEQUENCE LISTING (28 sequences)

SEQ ID NO: 1 - protein, 15 aa, Homo sapiens
MSARGEGAGQPSTSA

SEQ ID NO: 2 - DNA, 21 bp, Artificial
  PCR primer for amplification of HMGA2
gtgagccctctcctaagagac

SEQ ID NO: 3 - DNA, 20 bp, Artificial
  PCR primer for amplification of HMGA2
ctgcagtgtcttctcccttc

SEQ ID NO: 4 - DNA, 21 bp, Artificial
  PCR primer for amplification of GAPDH
gaccacagtccatgccatcac

SEQ ID NO: 5 - DNA, 22 bp, Artificial
  PCR primer for amplification of GAPDH
cataccaggaaatgagcttgac

SEQ ID NO: 6 - DNA, 21 bp, Artificial
  Oligonucleotide encoding HMGA2 siRNA
cagcaatctgtcgctaaggtt

SEQ ID NO: 7 - DNA, 21 bp, Artificial
  Oligonucleotide encoding HMGA2 siRNA
ccttagcgacagattgctgtt

SEQ ID NO: 8 - DNA, 21 bp, Artificial
  Oligonucleotide encoding scrambled siRNA
gagcgatcagatgatccactt

SEQ ID NO: 9 - DNA, 21 bp, Artificial
  Oligonucleotide encoding scrambled siRNA
gtggatcatctgatcgctctt

SEQ ID NO: 10 - DNA, 23 bp, Artificial
  PCR primer for RT-PCR of HMGA2 (forward)
tcccttcacagtcccaggtttag

SEQ ID NO: 11 - DNA, 20 bp, Artificial
  PCR primer for RT-PCR of HMGA2 (reverse)
tttttctcacccgcccactc

SEQ ID NO: 12 - DNA, 21 bp, Artificial
  PCR primer for RT-PCR of 18S rRNA (forward)
cgtctgccctatcaactttcg

SEQ ID NO: 13 - DNA, 22 bp, Artificial
  PCR primer for RT-PCR of 18S rRNA (reverse)
ggatgtggtagccgtttctcag

SEQ ID NO: 14 - DNA, 32 bp, Artificial
  Primer for generating HMGA2(2K/R) construct
  misc_feature (15)..(15): Mutation site
  misc_feature (18)..(18): Mutation site
gaaagcagctcaaaggagagcagagaccattg

SEQ ID NO: 15 - DNA, 30 bp, Artificial
  Primer for generating HMGA2(1-24 deletion) construct
ttactagaattccagaagcgaggacgcggc

SEQ ID NO: 16 - DNA, 30 bp, Artificial
  Primer for generating HMGA2(1-24 deletion) construct
cgcaggtctagactaatcttcctctgcgga

SEQ ID NO: 17 - DNA, 30 bp, Artificial
  Primer for generating HMGA2(83-109 deletion) construct
gagctcggatccgccgccatgggctaccca

SEQ ID NO: 18 - DNA, 30 bp, Artificial
  Primer for generating HMGA2(83-109 deletion) construct
gccagatctagactaccatttcctgggtct

SEQ ID NO: 19 - DNA, 30 bp, Artificial; identical to SEQ ID NO: 17
  Primer for generating HMGA2(66-109 deletion) construct

SEQ ID NO: 20 - DNA, 30 bp, Artificial
  Primer for generating HMGA2(66-109 deletion) construct
gccagatctagactaagctgctttcgaggg

SEQ ID NO: 21 - DNA, 30 bp, Artificial; identical to SEQ ID NO: 17
  Primer for generating HMGA2(42-109 deletion) construct

SEQ ID NO: 22 - DNA, 30 bp, Artificial
  Primer for generating HMGA2(42-109 deletion) construct
cgcaggtctagactaacaggttggctcttg

SEQ ID NO: 23 - DNA, 33 bp, Artificial
  Primer for generating HMGA2(4P/A) construct
  misc_feature (11)..(11): Mutation site
  misc_feature (23)..(23): Mutation site
tcctaagagagccagaggaagagccaaaggcag

SEQ ID NO: 24 - DNA, 33 bp, Artificial
  Primer for generating HMGA2(4P/A) construct
  misc_feature (11)..(11): Mutation site
  misc_feature (23)..(23): Mutation site
agaaaaacgggcaagaggcagagccaggaaatg

SEQ ID NO: 25 - DNA, 17 bp, Artificial
  PCR primer for RT-PCR of PUMA (forward)
gacctcaacgcacagta

SEQ ID NO: 26 - DNA, 17 bp, Artificial
  PCR primer for RT-PCR of PUMA (reverse)
ctaattgggctccatct

SEQ ID NO: 27 - DNA, 18 bp, Artificial
  PCR primer for RT-PCR of NOXA (forward)
actgttcgtgttcagctc

SEQ ID NO: 28 - DNA, 18 bp, Artificial
  PCR primer for RT-PCR of NOXA (reverse)
gtagcacactcgacttcc